SEQUENCE LISTING (63 sequences)

SEQ ID NO: 1 - DNA, 819 bp, Corynebacterium aurimicosum
ctaggcggcttcgccctcagcgggcttactatccaccacttccccatccacgtgctgcggatagtcaagggcggattcggataggtctacacgcactgtttcgtccgctgcgataccccgatctaggtcggtagatgacggcatccacttcgcgagctggcgtacacaggtcttgtgggccatagcgtcaaaattgtcagcccacgggccaaacacttcaccttgcttgttcttagccttggcgaattcatcacggtaggccagcatttcgtccttactcatgacgatgaagctgtgtcccccggtggtgaacttagctaccgcgtagtaggcgattgggttgcccttcggaccgttcatgcatggcttgtggacaagcttgtcatccagcccatagtcaacgtcaaagtggtcgttggcgtacacggtgcgggcgataagcgactgaatctgcccagagcggtgagccagctcaactaagccacggtaaccaaccacaagttgagccaccatgccgccctgcttcctatcccagaatggcagcaggtaagcatggcctagtacgccgggacgcaaaccaagctgggagcacgtcatgagtccacccagtacggactgtggggtgcattctgcaagcttcggggtttggcgcaggcaggtaagcgcatcacgtactagctgctgtgcctccatgccctttggcattgcgagctggaattgtgattccatgcttcggatttggtctgccagggtgacggggcgattctgcttagccggggcgttgttagcggccatacgctgctctagattgtttcccat

SEQ ID NO: 2 - protein, 272 aa, Corynebacterium aurimucosum
MGNNLEQRMAANNAPAKQNRPVTLADQIRSMESQFQLAMPKGMEAQQLVRDALTCLRQTPKLAECTPQSVLGGLMTCSQLGLRPGVLGHAYLLPFWDRKQGGMVAQLVVGYRGLVELAHRSGQIQSLIARTVYANDHFDVDYGLDDKLVHKPCMNGPKGNPIAYYAVAKFTTGGHSFIVMSKDEMLAYRDEFAKAKNKQGEVFGPWADNFDAMAHKTCVRQLAKWMPSSTDLDRGIAADETVRVDLSESALDYPQHVDGEVVDSKPAEGEAA

SEQ ID NO: 3 - DNA, 4100 bp, E.coli
ggtcgactctagaggatccccaaggagatatagatatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatgataccgagctcgaattcactggccgtcgttttacagccaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagatctatcgattttcgttcgtgaatacatgttataataactataactaataacgtaacgtgactggcaagagatatttttaaaacaatgaataggtttacacttactttagttttatggaaatgaaagatcatatcatatataatctagaataaaattaactaaaataattattatctagataaaaaatttagaagccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaattggtaatcagattttaga

SEQ ID NO: 4 - DNA, 3700 bp, E.coli
ggtcgactctagaggatccccaaggagatatacatatgactaagcaaccaccaatcgcaaaagccgatctgcaaaaaactcagggaaaccgtgcaccagcagcagttaaaaatagcgacgtgattagttttattaaccagccatcaatgaaagagcaactggcagcagctcttccacgccatatgacggctgaacgtatgatccgtatcgccaccacagaaattcgtaaagttccggcgttaggaaactgtgacactatgagttttgtcagtgcgatcgtacagtgttcacagctcggacttgagccaggtagcgccctcggtcatgcatatttactgccttttggtaataaaaacgaaaagagcggtaaaaagaacgttcagctaatcattggctatcgcggcatgattgatctggctcgccgttctggtcaaatcgccagcctgtcagcccgtgttgtccgtgaaggtgacgagtttagcttcgaatttggccttgatgaaaagttaatacaccgcccgggagaaaacgaagatgccccggttacccacgtctatgctgtcgcaagactgaaagacggaggtactcagtttgaagttatgacgcgcaaacagattgagctggtgcgcagcctgagtaaagctggtaataacgggccgtgggtaactcactgggaagaaatggcaaagaaaacggctattcgtcgcctgttcaaatatttgcccgtatcaattgagatccagcgtgcagtatcaatggatgaaaaggaaccactgacaatcgatcctgcagattcctctgtattaaccggggaatacagtgtaatcgataattcagaggaataagggtaccgagctcgaattcactggccgtcgttttacagccaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagatctatcgattttcgttcgtgaatacatgttataataactataactaataacgtaacgtgactggcaagagatatttttaaaacaatgaataggtttacacttactttagttttatggaaatgaaagatcatatcatatataatctagaataaaattaactaaaataattattatctagataaaaaatttagaagccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaa

SEQ ID NO: 5 - DNA, 3650 bp, Mycobacterium
ggtcgactctagaggatcccgatctaaggagatatacatatgacctcctcggagatcgacaagatcgatccggacgcaggcactcttgagtggcttcctgcggcacaggcgccttccgcagtctcacgcgcgatgttgcgagaacacgctcagatgatggcggacgcttaccaactcgcgaagggtatctgcggagatggacggaatggcactggtctgatggttccacagcgctttcgcggtaaacccgaagaagcggcagctgcaatgatttacggctctgagctcggtctgaacccacttcaggctgtgcaacgagtagtacccatccatggcatgccaaccctggaagctcggaccatggtgggactgcttaaagctcgcggttacaagatccgcaccgtcgaacagtcggacacttccgtcacagtcgaaggcgtcgcaccagacggcgaaacggctagctcaacctggaccatcgaacgcgcacaacgcgccggctacgttccaaccccatcatcaccggactccaagcgtcgacccgacgtcgacgatgactgggttaccgtgtcgaaaacatgggatggaaaggtcaagaagtccgttgttgggaacatgaagtacatcaccgacccacaggcgatgctgaaagctaaagcccagtctgaggtgtgtcgcgatctggcgcccgacgttctgatgggcatctcctatacccgggaggatctggaatctgaacgccaggatcagttcgaggatcgtcgtcccgcagcagccccagcacgcactcagcgtgtaaccgttgacgaaatcttcgccgaggaagtgccgttgtcctccgatggctttgccggtgataaccctccgacggcagctcaagctgcaccggagcctgccccggaacctgacacgcaccaagctcctgccggggatgtgatcgaggaccaaggcgatggtgcgccggatcccggtgaaccttccccagcggaacagagcgagacagttgtcgaaccagccccagagcccgcccctgcaccggataccgctgccgctgctaagtccacgtccaagaaagtgaacgctaaacgctctaccgcgccagcagctaaccctgacaaaccaaagacccgcatgcgtcaagctctggagaagcgcttgttcgcactcttgggcgatgccggacttagcggagcggccgaccgcgacggtcggattgccgtgtaccgttccgtgctcgaaaacgaggcaattggctccactgacgatctggatgaccctgcgattgggaaggtggcagatcagctctatgcatggcagcagcagaatgaactggatgacaagattgccggtatcctcagcgatgctgcccgtgctgagtctgagtcggtcgccccagcagcagatccgacttctgagggcaatgagtgaattcgtaccgagctcgaattcactggccgtcgttttacagccaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagatctatcgattttcgttcgtgaatacatgttataataactataactaataacgtaacgtgactggcaagagatatttt

SEQ ID NO: 6 - DNA, 3706 bp, Mycobacterium
taggagggtcgactctagaggatccccgatctaggagatatacatatggctgagaatgcggtaacaaagcaggacagcccgaaagcaccggaaaccatctctcaggttctccaagtcctggttcctcagttggctcgcgcagtgcctaaaggcatggacccagatcgcattgcacgcatcgtacagaccgaaattcgcaagtcacgcaacgcgaaagcggcaggcattgccaagcagtccttggacgattgcacccaagagagctttgcgggagctctcctcacttcggccgcactcggtttggaacctggagtgaacggcgaatgctacctggtgccctatcgcgatactcgacgcggcgtggtagagtgtcagctgatcatcgggtatcagggtatcgtcaagctgttttggcaacatccgcgcgcatcgcgcattgatgcgcagtgggttggcgcaaacgatgaattccactacaccatgggcctgaatcccacgctcaaacacgtcaaggcaaagggagatcggggcaatccagtctacttctacgcgatcgtggaagttaccggtgccgagcctctgtgggacgttttcaccgccgacgagatccgtgaactgcgtcgtgggaaggttggttcctccggcgatatcaaggatccacagcggtggatggaacgtaagactgccttgaaacaggtcctgaagcttgctcccaagacgacacgccttgacgctgctatccgagctgatgaccgccctggtaccgacctctctcagtcccaagcacttgctcttccctccacggtgaaaccgaccgctgactacatcgatggcgagattgccgaaccacacgaagtcgataccccaccgaagtcatcccgtgcccaacgagcacaacgcgcaactgccccagcaccagacgttcagatggcaaacccagaccagctgaaacggcttggtgagattcagaaggcggagaagtacaacgatgctgactggttcaagtttctggccgattctgcgggagtgaaggcaacccgtgctgccgatctgaccttcgatgaggctaaagctgtcatcgacatgttcgacggtcctaacgcgtaacaatttaccgagctcgaattcactggccgtcgttttacagccaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagatctatcgattttcgttcgtgaatacatgttataataactataactaataacgtaacgtgactggcaagagatatttttaaaacaatgaataggtttacacttactttagttttatggaaatgaaagatcatatcatatataatctagaataaaattaactaaaataattattatctagataaaaaatttagaagccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaa

SEQ ID NO: 7 - DNA, 3950 bp, Corynebacterium aurimucosum
ggtcgactctagaggatcccgatccaaggagatatacatatgggaaacaatctagagcagcgtatggccgctaacaacgccccggctaagcagaatcgccccgtcaccctggcagaccaaatccgaagcatggaatcacaattccagctcgcaatgccaaagggcatggaggcacagcagctagtacgtgatgcgcttacctgcctgcgccaaaccccgaagcttgcagaatgcaccccacagtccgtactgggtggactcatgacgtgctcccagcttggtttgcgtcccggcgtactaggccatgcttacctgctgccattctgggataggaagcagggcggcatggtggctcaacttgtggttggttaccgtggcttagttgagctggctcaccgctctgggcagattcagtcgcttatcgcccgcaccgtgtacgccaacgaccactttgacgttgactatgggctggatgacaagcttgtccacaagccatgcatgaacggtccgaagggcaacccaatcgcctactacgcggtagctaagttcaccaccgggggacacagcttcatcgtcatgagtaaggacgaaatgctggcctaccgtgatgaattcgccaaggctaagaacaagcaaggtgaagtgtttggcccgtgggctgacaattttgacgctatggcccacaagacctgtgtacgccagctcgcgaagtggatgccgtcatctaccgacctagatcggggtatcgcagcggacgaaacagtgcgtgtagacctatccgaatccgcccttgactatccgcagcacgtggatggggaagtggtggatagtaagcccgctgagggcgaagccgcctagcaattggtaccgagctcgaattcactggccgtcgttttacagccaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagatctatcgattttcgttcgtgaatacatgttataataactataactaataacgtaacgtgactggcaagagatatttttaaaacaatgaataggtttacacttactttagttttatggaaatgaaagatcatatcatatataatctagaataaaattaactaaaataattattatctagataaaaaatttagaagccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgtta

SEQ ID NO: 8 - DNA, 9137 bp, E.coli
aagcttgcatgcctgcaggtcgactctagaggatctaaggagatatacatatgactaagcaaccaccaatcgcaaaagccgatctgcaaaaaactcagggaaaccgtgcaccagcagcagttaaaaatagcgacgtgattagttttattaaccagccatcaatgaaagagcaactggcagcagctcttccacgccatatgacggctgaacgtatgatccgtatcgccaccacagaaattcgtaaagttccggcgttaggaaactgtgacactatgagttttgtcagtgcgatcgtacagtgttcacagctcggacttgagccaggtagcgccctcggtcatgcatatttactgccttttggtaataaaaacgaaaagagcggtaaaaagaacgttcagctaatcattggctatcgcggcatgattgatctggctcgccgttctggtcaaatcgccagcctgtcagcccgtgttgtccgtgaaggtgacgagtttagcttcgaatttggccttgatgaaaagttaatacaccgcccgggagaaaacgaagatgccccggttacccacgtctatgctgtcgcaagactgaaagacggaggtactcagtttgaagttatgacgcgcaaacagattgagctggtgcgcagcctgagtaaagctggtaataacgggccgtgggtaactcactgggaagaaatggcaaagaaaacggctattcgtcgcctgttcaaatatttgcccgtatcaattgagatccagcgtgcagtatcaatggatgaaaaggaaccactgacaatcgatcctgcagattcctctgtattaaccggggaatacagtgtaatcgataattcagaggaataattcagcctggaattcactggccgtcgttttacagccaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagatctatcgattttcgttcgtgaatacatgttataataactataactaataacgtaacgtgactggcaagagatatttttaaaacaatgaataggtttacacttactttagttttatggaaatgaaagatcatatcatatataatctagaataaaattaactaaaataattattatctagataaaaaatttagaagccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaattggtaatcagattttagaaaacaataaacccttgcatatgatatcgatgtacagatccctggtatgagtcagcaacaccttcttcacgaggcagacctcagcgcccccccccccctagcttgtctacgtctgatgctttgaatcggacggacttgccgatcttgtatgcggtgatttttccctcgtttgcccactttttaatggtggccggggtgagagctacgcgggcggcgacctgctgcgctgtgatccaatattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagttcctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctagattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttgacaatggctgatttcatcaggaatggaactgtcatgctgttatgtgcctggctcctaatcaaagctggggacaatgggttgccccgttgatctgatctagttcggattggcggggcttcactgtatctgggggtggcatcgtgaatagattgcacaccgtagtgggcagtgtgcacaccatagtggccatgagcaccaccacccccagggacgccgacggcgcgaagctctgcgcctggtgcggctcggagatcaagcaatccggcgtcggccggagccgggactactgccgccgctcctgccgccagcgggcgtacgaggcccggcgccagcgcgaggcgatcgtgtccgccgtggcgtcggcagtcgctcgccgagatacgtcacgtgacgaaatgcagcagccttccattccgtcacgtgacgaaactcgggccgcaggtcagagcacggttccgcccgctccggccctgccggacccccggcatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatggcctgtcgcttgcggtattcggaatcttgcacgccctcgctcaagccttcgtcactggtcccgccaccaaacgtttcggcgagaagcaggccattatcgccggcatggcggccgacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattaaaagatatgaccatgattacgcc

SEQ ID NO: 9 - DNA, 9100 bp, E.coli
aagcttgcatgcctgcaggtcgactctagaggatctaaggagatatacatatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatgaattcactggccgtcgttttacagccaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagatctatcgattttcgttcgtgaatacatgttataataactataactaataacgtaacgtgactggcaagagatatttttaaaacaatgaataggtttacacttactttagttttatggaaatgaaagatcatatcatatataatctagaataaaattaactaaaataattattatctagataaaaaatttagaagccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaattggtaatcagattttagaaaacaataaacccttgcatatgatatcgatgtacagatccctggtatgagtcagcaacaccttcttcacgaggcagacctcagcgcccccccccccctagcttgtctacgtctgatgctttgaatcggacggacttgccgatcttgtatgcggtgatttttccctcgtttgcccactttttaatggtggccggggtgagagctacgcgggcggcgacctgctgcgctgtgatccaatattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagttcctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctagattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttgacaatggctgatttcatcaggaatggaactgtcatgctgttatgtgcctggctcctaatcaaagctggggacaatgggttgccccgttgatctgatctagttcggattggcggggcttcactgtatctgggggtggcatcgtgaatagattgcacaccgtagtgggcagtgtgcacaccatagtggccatgagcaccaccacccccagggacgccgacggcgcgaagctctgcgcctggtgcggctcggagatcaagcaatccggcgtcggccggagccgggactactgccgccgctcctgccgccagcgggcgtacgaggcccggcgccagcgcgaggcgatcgtgtccgccgtggcgtcggcagtcgctcgccgagatacgtcacgtgacgaaatgcagcagccttccattccgtcacgtgacgaaactcgggccgcaggtcagagcacggttccgcccgctccggccctgccggacccccggcatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatggcctgtcgcttgcggtattcggaatcttgcacgccctcgctcaagccttcgtcactggtcccgccaccaaacgtttcggcgagaagcaggccattatcgccggcatggcggccgacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattaaaagatatgaccatgattacg

SEQ ID NO: 10 - DNA, 1429 bp, Mycobacterium
ctgcaggtcgaccggcagatctaaggagatatacatatgacctcctcggagatcgacaagatcgatccggacgcaggcactcttgagtggcttcctgcggcacaggcgccttccgcagtctcacgcgcgatgttgcgagaacacgctcagatgatggcggacgcttaccaactcgcgaagggtatctgcggagatggacggaatggcactggtctgatggttccacagcgctttcgcggtaaacccgaagaagcggcagctgcaatgatttacggctctgagctcggtctgaacccacttcaggctgtgcaacgagtagtacccatccatggcatgccaaccctggaagctcggaccatggtgggactgcttaaagctcgcggttacaagatccgcaccgtcgaacagtcggacacttccgtcacagtcgaaggcgtcgcaccagacggcgaaacggctagctcaacctggaccatcgaacgcgcacaacgcgccggctacgttccaaccccatcatcaccggactccaagcgtcgacccgacgtcgacgatgactgggttaccgtgtcgaaaacatgggatggaaaggtcaagaagtccgttgttgggaacatgaagtacatcaccgacccacaggcgatgctgaaagctaaagcccagtctgaggtgtgtcgcgatctggcgcccgacgttctgatgggcatctcctatacccgggaggatctggaatctgaacgccaggatcagttcgaggatcgtcgtcccgcagcagccccagcacgcactcagcgtgtaaccgttgacgaaatcttcgccgaggaagtgccgttgtcctccgatggctttgccggtgataaccctccgacggcagctcaagctgcaccggagcctgccccggaacctgacacgcaccaagctcctgccggggatgtgatcgaggaccaaggcgatggtgcgccggatcccggtgaaccttccccagcggaacagagcgagacagttgtcgaaccagccccagagcccgcccctgcaccggataccgctgccgctgctaagtccacgtccaagaaagtgaacgctaaacgctctaccgcgccagcagctaaccctgacaaaccaaagacccgcatgcgtcaagctctggagaagcgcttgttcgcactcttgggcgatgccggacttagcggagcggccgaccgcgacggtcggattgccgtgtaccgttccgtgctcgaaaacgaggcaattggctccactgacgatctggatgaccctgcgattgggaaggtggcagatcagctctatgcatggcagcagcagaatgaactggatgacaagattgccggtatcctcagcgatgctgcccgtgctgagtctgagtcggtcgccccagcagcagatccgacttctgagggcaatgagtgaattc

SEQ ID NO: 11 - DNA, 1093 bp, Mycobacterium
gcagatctaaggagatatacatatggctgagaatgcggtaacaaagcaggacagcccgaaagcaccggaaaccatctctcaggttctccaagtcctggttcctcagttggctcgcgcagtgcctaaaggcatggacccagatcgcattgcacgcatcgtacagaccgaaattcgcaagtcacgcaacgcgaaagcggcaggcattgccaagcagtccttggacgattgcacccaagagagctttgcgggagctctcctcacttcggccgcactcggtttggaacctggagtgaacggcgaatgctacctggtgccctatcgcgatactcgacgcggcgtggtagagtgtcagctgatcatcgggtatcagggtatcgtcaagctgttttggcaacatccgcgcgcatcgcgcattgatgcgcagtgggttggcgcaaacgatgaattccactacaccatgggcctgaatcccacgctcaaacacgtcaaggcaaagggagatcggggcaatccagtctacttctacgcgatcgtggaagttaccggtgccgagcctctgtgggacgttttcaccgccgacgagatccgtgaactgcgtcgtgggaaggttggttcctccggcgatatcaaggatccacagcggtggatggaacgtaagactgccttgaaacaggtcctgaagcttgctcccaagacgacacgccttgacgctgctatccgagctgatgaccgccctggtaccgacctctctcagtcccaagcacttgctcttccctccacggtgaaaccgaccgctgactacatcgatggcgagattgccgaaccacacgaagtcgataccccaccgaagtcatcccgtgcccaacgagcacaacgcgcaactgccccagcaccagacgttcagatggcaaacccagaccagctgaaacggcttggtgagattcagaaggcggagaagtacaacgatgctgactggttcaagtttctggccgattctgcgggagtgaaggcaacccgtgctgccgatctgaccttcgatgaggctaaagctgtcatcgacatgttcgacggtcctaacgcgtaacaattgata

SEQ ID NO: 12 - DNA, 100 bp, artificial
  Gene
aacaacgatgactgggcgggtctctcctgcttcgttgagcacctcaagtcaagctgcgtcagtcaaaatggccacagctttcgcagcgttatccgtacct

SEQ ID NO: 13 - DNA, 100 bp, artificial
  Gene
aacaacgatgactgggcgggtctctcctgcttcgttgagcacctcaagtgcagcggcgtcagtcaaaatggccacagctttcgcagcgttatccgtacct

SEQ ID NO: 14 - DNA, 100 bp, artificial
  Gene
aacaacgatgactgggcgggtctctcctgcttcgttgagcacctcaaggcaagctgcgtcagtcaaaatggccacagctttcgcagcgttatccgtacct

SEQ ID NO: 15 - DNA, 100 bp, artificial; identical to SEQ ID NO: 13
  Gene

SEQ ID NO: 16 - DNA, 100 bp, artificial
  Gene
aacaacgatgactgggcgggtctctcctgcttcgttgagcacctcaagttcagcggcgtcagtcaaaatggccacagctttcgcagcgttatccgtacct

SEQ ID NO: 17 - DNA, 100 bp, artificial
  Gene
aacaacgatgactgggcgggtctctcctgcttcgttgagcacctcaaggaaagctgcgtcagtcaaaatggccacagctttcgcagcgttatccgtacct

SEQ ID NO: 18 - DNA, 100 bp, artificial
  Gene
aacaacgatgactgggcgggtctctcctgcttcgttgagcacctcaaggtgagctgcgtcagtcaaaatggccacagctttcgcagcgttatccgtacct

SEQ ID NO: 19 - DNA, 100 bp, artificial
  Gene
aacaacgatgactgggcgggtctctcctgcttcgttgagcacctcaaggatagctgcgtcagtcaaaatggccacagctttcgcagcgttatccgtacct

SEQ ID NO: 20 - DNA, 100 bp, artificial
  Gene
aacaacgatgactgggcgggtctctcctgcttcgttgagcacctcaagcttagctgcgtcagtcaaaatggccacagctttcgcagcgttatccgtacct

SEQ ID NO: 21 - DNA, 100 bp, artificial
  Gene
aacaacgatgactgggcgggtctctcctgcttcgttgagcacctcaagcagagctgcgtcagtcaaaatggccacagctttcgcagcgttatccgtacct

SEQ ID NO: 22 - DNA, 100 bp, artificial
  Gene
aacaacgatgactgggcgggtctctcctgcttcgttgagcacctcaagcatagctgcgtcagtcaaaatggccacagctttcgcagcgttatccgtacct

SEQ ID NO: 23 - DNA, 100 bp, artificial
  Gene
aacaacgatgactgggcgggtctctcctgcttcgttgagcacctcaaggttagctgcgtcagtcaaaatggccacagctttcgcagcgttatccgtacct

SEQ ID NO: 24 - DNA, 100 bp, artificial
  Gene
aacaacgatgactgggcgggtctctcctgcttcgttgagcacctcaagtggagctgcgtcagtcaaaatggccacagctttcgcagcgttatccgtacct

SEQ ID NO: 25 - DNA, 100 bp, artificial
  Gene
aacaacgatgactgggcgggtctctcctgcttcgttgagcacctcaagctgagctgcgtcagtcaaaatggccacagctttcgcagcgttatccgtacct

SEQ ID NO: 26 - DNA, 100 bp, artificial
  Gene
aacaacgatgactgggcgggtctctcctgcttcgttgagcacctcaaggcgagctgcgtcagtcaaaatggccacagctttcgcagcgttatccgtacct

SEQ ID NO: 27 - DNA, 100 bp, artificial
  Gene
aacaacgatgactgggcgggtctctcctgcttcgttgagcacctcaagggaagctgcgtcagtcaaaatggccacagctttcgcagcgttatccgtacct

SEQ ID NO: 28 - DNA, 100 bp, artificial
  Gene
aacaacgatgactgggcgggtctctcctgcttcgttgagcacctcaagggtagctgcgtcagtcaaaatggccacagctttcgcagcgttatccgtacct

SEQ ID NO: 29 - DNA, 100 bp, artificial
  Gene
aacaacgatgactgggcgggtctctcctgcttcgttgagcacctcaagcacagcggcgtcagtcaaaatggccacagctttcgcagcgttatccgtacct

SEQ ID NO: 30 - DNA, 100 bp, artificial
  Gene
aacaacgatgactgggcgggtctctcctgcttcgttgagcacctcaagccaagctgcgtcagtcaaaatggccacagctttcgcagcgttatccgtacct

SEQ ID NO: 31 - DNA, 100 bp, artificial
  Gene
aacaacgatgactgggcgggtctctcctgcttcgttgagcacctcaaggtaagctgcgtcagtcaaaatggccacagctttcgcagcgttatccgtacct

SEQ ID NO: 32 - DNA, 100 bp, artificial
  Gene
tcttcaagatctccatcgcgcggcggccgtcggaacgagggcaggtgaagatgatatcggtggtgccgtcttctacagaagagacgttctgcagaaccat

SEQ ID NO: 33 - DNA, 1566 bp, Corynebacterium glutamicum
atggcaaccacgttgctggacctcaccaaacttatcgatggcatcctcaagggctctgcccagggcgttcccgctcacgcagtaggggaacaagcaatcgcggctattggtcttgactcctccagcttacctacctcggacgctatttttgctgcagttccaggaacccgcactcacggcgcacagtttgcaggtacggataacgctgcgaaagctgtggccattttgactgacgcagcttggcttgaggtgctcaacgaagcaggagagacccgcccagtcatcgttgttgatgatgtccgcgcagtacttggcgcagcatcatcaagcatttatggcgatccttcaaaagatttcacgctcattggagtcactggaacctcaggtaaaaccaccaccagctacctcttggaaaaaggactcatggaggcaggccacaaagttggtttgatcggcaccacaggtacacgtattgacggggaagaagtacccacaaagctcaccactccagaagcgccgactctgcaggcattgtttgctcgaatgcgcgatcacggtgtcacccacgtggtgatggaagtatccagccatgcattgtcattgggcagagttgcgggttcccactttgatgtagctgcgtttaccaacctgtcgcaggatcaccttgatttccaccccaccatggatgattactttgacgcgaaggcattgttcttccgcgcagattctccacttgtggctgacaaacaggtcgtgtgcgtggatgattcttggggtcagcgcatggccagcgtggcagcggatgtgcaaacagtatccacccttgggcaagaagcagacttcagcgctacagacatcaatgtcagcgactctggcgcccagagttttaagatcaacgccccctcaaaccagtcctaccaggtcgagctagctcttccaggtgcgttcaacgttgctaacgccacgttggcatttgccgctgcggcacgcgtgggtgttgatggcgaagcgtttgctcgaggcatgtccaaggtcgcggttccaggccgtatggaacgcattgatgagggacaagacttccttgcagtggtggattatgcccacaagcctgctgcagtggctgctgtgttggatacgttgaggacccagattgacgggcgcctcggagtggttatcggtgctggtggagaccgcgattccaccaagcgtggccccatggggcagttgtccgcacagcgtgctgatctagttattgtcactgatgacaaccctcgttcagaggtgcctgccacgattcgcgcagcagtcactgcaggagcacagcagggtgcttcagagtccgaacgaccggtggaagtcctagaaattggtgaccgtgcagaagcaattcgcgttttggtcgagtgggcacagcctggagatggcattgtagtagctggaaaaggccatgaagttggacaactagttgctggtgtcacccaccattttgatgaccgcgaagaagttcgcgctgctttgacagaaaagctcaacaataaacttccccttactacggaagaaggatag

SEQ ID NO: 34 - DNA, 1566 bp, Corynebacterium glutamicum
atggcaaccacgttgctggacctcaccaaacttatcgatggcatcctcaagggctctgcccagggcgttcccgctcacgcagtaggggaacaagcaatcgcggctattggtcttgactcctccagcttacctacctcggacgctatttttgctgcagttccaggaacccgcactcacggcgcacagtttgcaggtacggataacgctgcgaaagctgtggccattttgactgacgcagcttaccttgaggtgctcaacgaagcaggagagacccgcccagtcatcgttgttgatgatgtccgcgcagtacttggcgcagcatcatcaagcatttatggcgatccttcaaaagatttcacgctcattggagtcactggaacctcaggtaaaaccaccaccagctacctcttggaaaaaggactcatggaggcaggccacaaagttggtttgatcggcaccacaggtacacgtattgacggggaagaagtacccacaaagctcaccactccagaagcgccgactctgcaggcattgtttgctcgaatgcgcgatcacggtgtcacccacgtggtgatggaagtatccagccatgcattgtcattgggcagagttgcgggttcccactttgatgtagctgcgtttaccaacctgtcgcaggatcaccttgatttccaccccaccatggatgattactttgacgcgaaggcattgttcttccgcgcagattctccacttgtggctgacaaacaggtcgtgtgcgtggatgattcttggggtcagcgcatggccagcgtggcagcggatgtgcaaacagtatccacccttgggcaagaagcagacttcagcgctacagacatcaatgtcagcgactctggcgcccagagttttaagatcaacgccccctcaaaccagtcctaccaggtcgagctagctcttccaggtgcgttcaacgttgctaacgccacgttggcatttgccgctgcggcacgcgtgggtgttgatggcgaagcgtttgctcgaggcatgtccaaggtcgcggttccaggccgtatggaacgcattgatgagggacaagacttccttgcagtggtggattatgcccacaagcctgctgcagtggctgctgtgttggatacgttgaggacccagattgacgggcgcctcggagtggttatcggtgctggtggagaccgcgattccaccaagcgtggccccatggggcagttgtccgcacagcgtgctgatctagttattgtcactgatgacaaccctcgttcagaggtgcctgccacgattcgcgcagcagtcactgcaggagcacagcagggtgcttcagagtccgaacgaccggtggaagtcctagaaattggtgaccgtgcagaagcaattcgcgttttggtcgagtgggcacagcctggagatggcattgtagtagctggaaaaggccatgaagttggacaactagttgctggtgtcacccaccattttgatgaccgcgaagaagttcgcgctgctttgacagaaaagctcaacaataaacttccccttactacggaagaaggatag

SEQ ID NO: 35 - DNA, 1566 bp, Corynebacterium glutamicum
atggcaaccacgttgctggacctcaccaaacttatcgatggcatcctcaagggctctgcccagggcgttcccgctcacgcagtaggggaacaagcaatcgcggctattggtcttgactcctccagcttacctacctcggacgctatttttgctgcagttccaggaacccgcactcacggcgcacagtttgcaggtacggataacgctgcgaaagctgtggccattttgactgacgcagctaaccttgaggtgctcaacgaagcaggagagacccgcccagtcatcgttgttgatgatgtccgcgcagtacttggcgcagcatcatcaagcatttatggcgatccttcaaaagatttcacgctcattggagtcactggaacctcaggtaaaaccaccaccagctacctcttggaaaaaggactcatggaggcaggccacaaagttggtttgatcggcaccacaggtacacgtattgacggggaagaagtacccacaaagctcaccactccagaagcgccgactctgcaggcattgtttgctcgaatgcgcgatcacggtgtcacccacgtggtgatggaagtatccagccatgcattgtcattgggcagagttgcgggttcccactttgatgtagctgcgtttaccaacctgtcgcaggatcaccttgatttccaccccaccatggatgattactttgacgcgaaggcattgttcttccgcgcagattctccacttgtggctgacaaacaggtcgtgtgcgtggatgattcttggggtcagcgcatggccagcgtggcagcggatgtgcaaacagtatccacccttgggcaagaagcagacttcagcgctacagacatcaatgtcagcgactctggcgcccagagttttaagatcaacgccccctcaaaccagtcctaccaggtcgagctagctcttccaggtgcgttcaacgttgctaacgccacgttggcatttgccgctgcggcacgcgtgggtgttgatggcgaagcgtttgctcgaggcatgtccaaggtcgcggttccaggccgtatggaacgcattgatgagggacaagacttccttgcagtggtggattatgcccacaagcctgctgcagtggctgctgtgttggatacgttgaggacccagattgacgggcgcctcggagtggttatcggtgctggtggagaccgcgattccaccaagcgtggccccatggggcagttgtccgcacagcgtgctgatctagttattgtcactgatgacaaccctcgttcagaggtgcctgccacgattcgcgcagcagtcactgcaggagcacagcagggtgcttcagagtccgaacgaccggtggaagtcctagaaattggtgaccgtgcagaagcaattcgcgttttggtcgagtgggcacagcctggagatggcattgtagtagctggaaaaggccatgaagttggacaactagttgctggtgtcacccaccattttgatgaccgcgaagaagttcgcgctgctttgacagaaaagctcaacaataaacttccccttactacggaagaaggatag

SEQ ID NO: 36 - DNA, 1566 bp, Corynebacterium glutamicum
atggcaaccacgttgctggacctcaccaaacttatcgatggcatcctcaagggctctgcccagggcgttcccgctcacgcagtaggggaacaagcaatcgcggctattggtcttgactcctccagcttacctacctcggacgctatttttgctgcagttccaggaacccgcactcacggcgcacagtttgcaggtacggataacgctgcgaaagctgtggccattttgactgacgcagcttgccttgaggtgctcaacgaagcaggagagacccgcccagtcatcgttgttgatgatgtccgcgcagtacttggcgcagcatcatcaagcatttatggcgatccttcaaaagatttcacgctcattggagtcactggaacctcaggtaaaaccaccaccagctacctcttggaaaaaggactcatggaggcaggccacaaagttggtttgatcggcaccacaggtacacgtattgacggggaagaagtacccacaaagctcaccactccagaagcgccgactctgcaggcattgtttgctcgaatgcgcgatcacggtgtcacccacgtggtgatggaagtatccagccatgcattgtcattgggcagagttgcgggttcccactttgatgtagctgcgtttaccaacctgtcgcaggatcaccttgatttccaccccaccatggatgattactttgacgcgaaggcattgttcttccgcgcagattctccacttgtggctgacaaacaggtcgtgtgcgtggatgattcttggggtcagcgcatggccagcgtggcagcggatgtgcaaacagtatccacccttgggcaagaagcagacttcagcgctacagacatcaatgtcagcgactctggcgcccagagttttaagatcaacgccccctcaaaccagtcctaccaggtcgagctagctcttccaggtgcgttcaacgttgctaacgccacgttggcatttgccgctgcggcacgcgtgggtgttgatggcgaagcgtttgctcgaggcatgtccaaggtcgcggttccaggccgtatggaacgcattgatgagggacaagacttccttgcagtggtggattatgcccacaagcctgctgcagtggctgctgtgttggatacgttgaggacccagattgacgggcgcctcggagtggttatcggtgctggtggagaccgcgattccaccaagcgtggccccatggggcagttgtccgcacagcgtgctgatctagttattgtcactgatgacaaccctcgttcagaggtgcctgccacgattcgcgcagcagtcactgcaggagcacagcagggtgcttcagagtccgaacgaccggtggaagtcctagaaattggtgaccgtgcagaagcaattcgcgttttggtcgagtgggcacagcctggagatggcattgtagtagctggaaaaggccatgaagttggacaactagttgctggtgtcacccaccattttgatgaccgcgaagaagttcgcgctgctttgacagaaaagctcaacaataaacttccccttactacggaagaaggatag

SEQ ID NO: 37 - DNA, 1566 bp, Corynebacterium glutamicum
atggcaaccacgttgctggacctcaccaaacttatcgatggcatcctcaagggctctgcccagggcgttcccgctcacgcagtaggggaacaagcaatcgcggctattggtcttgactcctccagcttacctacctcggacgctatttttgctgcagttccaggaacccgcactcacggcgcacagtttgcaggtacggataacgctgcgaaagctgtggccattttgactgacgcagcttcccttgaggtgctcaacgaagcaggagagacccgcccagtcatcgttgttgatgatgtccgcgcagtacttggcgcagcatcatcaagcatttatggcgatccttcaaaagatttcacgctcattggagtcactggaacctcaggtaaaaccaccaccagctacctcttggaaaaaggactcatggaggcaggccacaaagttggtttgatcggcaccacaggtacacgtattgacggggaagaagtacccacaaagctcaccactccagaagcgccgactctgcaggcattgtttgctcgaatgcgcgatcacggtgtcacccacgtggtgatggaagtatccagccatgcattgtcattgggcagagttgcgggttcccactttgatgtagctgcgtttaccaacctgtcgcaggatcaccttgatttccaccccaccatggatgattactttgacgcgaaggcattgttcttccgcgcagattctccacttgtggctgacaaacaggtcgtgtgcgtggatgattcttggggtcagcgcatggccagcgtggcagcggatgtgcaaacagtatccacccttgggcaagaagcagacttcagcgctacagacatcaatgtcagcgactctggcgcccagagttttaagatcaacgccccctcaaaccagtcctaccaggtcgagctagctcttccaggtgcgttcaacgttgctaacgccacgttggcatttgccgctgcggcacgcgtgggtgttgatggcgaagcgtttgctcgaggcatgtccaaggtcgcggttccaggccgtatggaacgcattgatgagggacaagacttccttgcagtggtggattatgcccacaagcctgctgcagtggctgctgtgttggatacgttgaggacccagattgacgggcgcctcggagtggttatcggtgctggtggagaccgcgattccaccaagcgtggccccatggggcagttgtccgcacagcgtgctgatctagttattgtcactgatgacaaccctcgttcagaggtgcctgccacgattcgcgcagcagtcactgcaggagcacagcagggtgcttcagagtccgaacgaccggtggaagtcctagaaattggtgaccgtgcagaagcaattcgcgttttggtcgagtgggcacagcctggagatggcattgtagtagctggaaaaggccatgaagttggacaactagttgctggtgtcacccaccattttgatgaccgcgaagaagttcgcgctgctttgacagaaaagctcaacaataaacttccccttactacggaagaaggatag

SEQ ID NO: 38 - DNA, 1566 bp, Corynebacterium glutamicum
atggcaaccacgttgctggacctcaccaaacttatcgatggcatcctcaagggctctgcccagggcgttcccgctcacgcagtaggggaacaagcaatcgcggctattggtcttgactcctccagcttacctacctcggacgctatttttgctgcagttccaggaacccgcactcacggcgcacagtttgcaggtacggataacgctgcgaaagctgtggccattttgactgacgcagctttccttgaggtgctcaacgaagcaggagagacccgcccagtcatcgttgttgatgatgtccgcgcagtacttggcgcagcatcatcaagcatttatggcgatccttcaaaagatttcacgctcattggagtcactggaacctcaggtaaaaccaccaccagctacctcttggaaaaaggactcatggaggcaggccacaaagttggtttgatcggcaccacaggtacacgtattgacggggaagaagtacccacaaagctcaccactccagaagcgccgactctgcaggcattgtttgctcgaatgcgcgatcacggtgtcacccacgtggtgatggaagtatccagccatgcattgtcattgggcagagttgcgggttcccactttgatgtagctgcgtttaccaacctgtcgcaggatcaccttgatttccaccccaccatggatgattactttgacgcgaaggcattgttcttccgcgcagattctccacttgtggctgacaaacaggtcgtgtgcgtggatgattcttggggtcagcgcatggccagcgtggcagcggatgtgcaaacagtatccacccttgggcaagaagcagacttcagcgctacagacatcaatgtcagcgactctggcgcccagagttttaagatcaacgccccctcaaaccagtcctaccaggtcgagctagctcttccaggtgcgttcaacgttgctaacgccacgttggcatttgccgctgcggcacgcgtgggtgttgatggcgaagcgtttgctcgaggcatgtccaaggtcgcggttccaggccgtatggaacgcattgatgagggacaagacttccttgcagtggtggattatgcccacaagcctgctgcagtggctgctgtgttggatacgttgaggacccagattgacgggcgcctcggagtggttatcggtgctggtggagaccgcgattccaccaagcgtggccccatggggcagttgtccgcacagcgtgctgatctagttattgtcactgatgacaaccctcgttcagaggtgcctgccacgattcgcgcagcagtcactgcaggagcacagcagggtgcttcagagtccgaacgaccggtggaagtcctagaaattggtgaccgtgcagaagcaattcgcgttttggtcgagtgggcacagcctggagatggcattgtagtagctggaaaaggccatgaagttggacaactagttgctggtgtcacccaccattttgatgaccgcgaagaagttcgcgctgctttgacagaaaagctcaacaataaacttccccttactacggaagaaggatag

SEQ ID NO: 39 - DNA, 1566 bp, Corynebacterium glutamicum
atggcaaccacgttgctggacctcaccaaacttatcgatggcatcctcaagggctctgcccagggcgttcccgctcacgcagtaggggaacaagcaatcgcggctattggtcttgactcctccagcttacctacctcggacgctatttttgctgcagttccaggaacccgcactcacggcgcacagtttgcaggtacggataacgctgcgaaagctgtggccattttgactgacgccgctgtgcttgaggtgctcaacgaagcaggagagacccgcccagtcatcgttgttgatgatgtccgcgcagtacttggcgcagcatcatcaagcatttatggcgatccttcaaaagatttcacgctcattggagtcactggaacctcaggtaaaaccaccaccagctacctcttggaaaaaggactcatggaggcaggccacaaagttggtttgatcggcaccacaggtacacgtattgacggggaagaagtacccacaaagctcaccactccagaagcgccgactctgcaggcattgtttgctcgaatgcgcgatcacggtgtcacccacgtggtgatggaagtatccagccatgcattgtcattgggcagagttgcgggttcccactttgatgtagctgcgtttaccaacctgtcgcaggatcaccttgatttccaccccaccatggatgattactttgacgcgaaggcattgttcttccgcgcagattctccacttgtggctgacaaacaggtcgtgtgcgtggatgattcttggggtcagcgcatggccagcgtggcagcggatgtgcaaacagtatccacccttgggcaagaagcagacttcagcgctacagacatcaatgtcagcgactctggcgcccagagttttaagatcaacgccccctcaaaccagtcctaccaggtcgagctagctcttccaggtgcgttcaacgttgctaacgccacgttggcatttgccgctgcggcacgcgtgggtgttgatggcgaagcgtttgctcgaggcatgtccaaggtcgcggttccaggccgtatggaacgcattgatgagggacaagacttccttgcagtggtggattatgcccacaagcctgctgcagtggctgctgtgttggatacgttgaggacccagattgacgggcgcctcggagtggttatcggtgctggtggagaccgcgattccaccaagcgtggccccatggggcagttgtccgcacagcgtgctgatctagttattgtcactgatgacaaccctcgttcagaggtgcctgccacgattcgcgcagcagtcactgcaggagcacagcagggtgcttcagagtccgaacgaccggtggaagtcctagaaattggtgaccgtgcagaagcaattcgcgttttggtcgagtgggcacagcctggagatggcattgtagtagctggaaaaggccatgaagttggacaactagttgctggtgtcacccaccattttgatgaccgcgaagaagttcgcgctgctttgacagaaaagctcaacaataaacttccccttactacggaagaaggatag

SEQ ID NO: 40 - DNA, 1566 bp, Corynebacterium glutamicum
atggcaaccacgttgctggacctcaccaaacttatcgatggcatcctcaagggctctgcccagggcgttcccgctcacgcagtaggggaacaagcaatcgcggctattggtcttgactcctccagcttacctacctcggacgctatttttgctgcagttccaggaacccgcactcacggcgcacagtttgcaggtacggataacgctgcgaaagctgtggccattttgactgacgcagctctgcttgaggtgctcaacgaagcaggagagacccgcccagtcatcgttgttgatgatgtccgcgcagtacttggcgcagcatcatcaagcatttatggcgatccttcaaaagatttcacgctcattggagtcactggaacctcaggtaaaaccaccaccagctacctcttggaaaaaggactcatggaggcaggccacaaagttggtttgatcggcaccacaggtacacgtattgacggggaagaagtacccacaaagctcaccactccagaagcgccgactctgcaggcattgtttgctcgaatgcgcgatcacggtgtcacccacgtggtgatggaagtatccagccatgcattgtcattgggcagagttgcgggttcccactttgatgtagctgcgtttaccaacctgtcgcaggatcaccttgatttccaccccaccatggatgattactttgacgcgaaggcattgttcttccgcgcagattctccacttgtggctgacaaacaggtcgtgtgcgtggatgattcttggggtcagcgcatggccagcgtggcagcggatgtgcaaacagtatccacccttgggcaagaagcagacttcagcgctacagacatcaatgtcagcgactctggcgcccagagttttaagatcaacgccccctcaaaccagtcctaccaggtcgagctagctcttccaggtgcgttcaacgttgctaacgccacgttggcatttgccgctgcggcacgcgtgggtgttgatggcgaagcgtttgctcgaggcatgtccaaggtcgcggttccaggccgtatggaacgcattgatgagggacaagacttccttgcagtggtggattatgcccacaagcctgctgcagtggctgctgtgttggatacgttgaggacccagattgacgggcgcctcggagtggttatcggtgctggtggagaccgcgattccaccaagcgtggccccatggggcagttgtccgcacagcgtgctgatctagttattgtcactgatgacaaccctcgttcagaggtgcctgccacgattcgcgcagcagtcactgcaggagcacagcagggtgcttcagagtccgaacgaccggtggaagtcctagaaattggtgaccgtgcagaagcaattcgcgttttggtcgagtgggcacagcctggagatggcattgtagtagctggaaaaggccatgaagttggacaactagttgctggtgtcacccaccattttgatgaccgcgaagaagttcgcgctgctttgacagaaaagctcaacaataaacttccccttactacggaagaaggatag

SEQ ID NO: 41 - DNA, 1566 bp, Corynebacterium glutamicum
atggcaaccacgttgctggacctcaccaaacttatcgatggcatcctcaagggctctgcccagggcgttcccgctcacgcagtaggggaacaagcaatcgcggctattggtcttgactcctccagcttacctacctcggacgctatttttgctgcagttccaggaacccgcactcacggcgcacagtttgcaggtacggataacgctgcgaaagctgtggccattttgactgacgcagctcaccttgaggtgctcaacgaagcaggagagacccgcccagtcatcgttgttgatgatgtccgcgcagtacttggcgcagcatcatcaagcatttatggcgatccttcaaaagatttcacgctcattggagtcactggaacctcaggtaaaaccaccaccagctacctcttggaaaaaggactcatggaggcaggccacaaagttggtttgatcggcaccacaggtacacgtattgacggggaagaagtacccacaaagctcaccactccagaagcgccgactctgcaggcattgtttgctcgaatgcgcgatcacggtgtcacccacgtggtgatggaagtatccagccatgcattgtcattgggcagagttgcgggttcccactttgatgtagctgcgtttaccaacctgtcgcaggatcaccttgatttccaccccaccatggatgattactttgacgcgaaggcattgttcttccgcgcagattctccacttgtggctgacaaacaggtcgtgtgcgtggatgattcttggggtcagcgcatggccagcgtggcagcggatgtgcaaacagtatccacccttgggcaagaagcagacttcagcgctacagacatcaatgtcagcgactctggcgcccagagttttaagatcaacgccccctcaaaccagtcctaccaggtcgagctagctcttccaggtgcgttcaacgttgctaacgccacgttggcatttgccgctgcggcacgcgtgggtgttgatggcgaagcgtttgctcgaggcatgtccaaggtcgcggttccaggccgtatggaacgcattgatgagggacaagacttccttgcagtggtggattatgcccacaagcctgctgcagtggctgctgtgttggatacgttgaggacccagattgacgggcgcctcggagtggttatcggtgctggtggagaccgcgattccaccaagcgtggccccatggggcagttgtccgcacagcgtgctgatctagttattgtcactgatgacaaccctcgttcagaggtgcctgccacgattcgcgcagcagtcactgcaggagcacagcagggtgcttcagagtccgaacgaccggtggaagtcctagaaattggtgaccgtgcagaagcaattcgcgttttggtcgagtgggcacagcctggagatggcattgtagtagctggaaaaggccatgaagttggacaactagttgctggtgtcacccaccattttgatgaccgcgaagaagttcgcgctgctttgacagaaaagctcaacaataaacttccccttactacggaagaaggatag

SEQ ID NO: 42 - DNA, 1566 bp, Corynebacterium glutamicum
atggcaaccacgttgctggacctcaccaaacttatcgatggcatcctcaagggctctgcccagggcgttcccgctcacgcagtaggggaacaagcaatcgcggctattggtcttgactcctccagcttacctacctcggacgctatttttgctgcagttccaggaacccgcactcacggcgcacagtttgcaggtacggataacgctgcgaaagctgtggccattttgactgacgcagctatccttgaggtgctcaacgaagcaggagagacccgcccagtcatcgttgttgatgatgtccgcgcagtacttggcgcagcatcatcaagcatttatggcgatccttcaaaagatttcacgctcattggagtcactggaacctcaggtaaaaccaccaccagctacctcttggaaaaaggactcatggaggcaggccacaaagttggtttgatcggcaccacaggtacacgtattgacggggaagaagtacccacaaagctcaccactccagaagcgccgactctgcaggcattgtttgctcgaatgcgcgatcacggtgtcacccacgtggtgatggaagtatccagccatgcattgtcattgggcagagttgcgggttcccactttgatgtagctgcgtttaccaacctgtcgcaggatcaccttgatttccaccccaccatggatgattactttgacgcgaaggcattgttcttccgcgcagattctccacttgtggctgacaaacaggtcgtgtgcgtggatgattcttggggtcagcgcatggccagcgtggcagcggatgtgcaaacagtatccacccttgggcaagaagcagacttcagcgctacagacatcaatgtcagcgactctggcgcccagagttttaagatcaacgccccctcaaaccagtcctaccaggtcgagctagctcttccaggtgcgttcaacgttgctaacgccacgttggcatttgccgctgcggcacgcgtgggtgttgatggcgaagcgtttgctcgaggcatgtccaaggtcgcggttccaggccgtatggaacgcattgatgagggacaagacttccttgcagtggtggattatgcccacaagcctgctgcagtggctgctgtgttggatacgttgaggacccagattgacgggcgcctcggagtggttatcggtgctggtggagaccgcgattccaccaagcgtggccccatggggcagttgtccgcacagcgtgctgatctagttattgtcactgatgacaaccctcgttcagaggtgcctgccacgattcgcgcagcagtcactgcaggagcacagcagggtgcttcagagtccgaacgaccggtggaagtcctagaaattggtgaccgtgcagaagcaattcgcgttttggtcgagtgggcacagcctggagatggcattgtagtagctggaaaaggccatgaagttggacaactagttgctggtgtcacccaccattttgatgaccgcgaagaagttcgcgctgctttgacagaaaagctcaacaataaacttccccttactacggaagaaggatag

SEQ ID NO: 43 - DNA, 1566 bp, Corynebacterium glutamicum
atggcaaccacgttgctggacctcaccaaacttatcgatggcatcctcaagggctctgcccagggcgttcccgctcacgcagtaggggaacaagcaatcgcggctattggtcttgactcctccagcttacctacctcggacgctatttttgctgcagttccaggaacccgcactcacggcgcacagtttgcaggtacggataacgctgcgaaagctgtggccattttgactgacgcagctacccttgaggtgctcaacgaagcaggagagacccgcccagtcatcgttgttgatgatgtccgcgcagtacttggcgcagcatcatcaagcatttatggcgatccttcaaaagatttcacgctcattggagtcactggaacctcaggtaaaaccaccaccagctacctcttggaaaaaggactcatggaggcaggccacaaagttggtttgatcggcaccacaggtacacgtattgacggggaagaagtacccacaaagctcaccactccagaagcgccgactctgcaggcattgtttgctcgaatgcgcgatcacggtgtcacccacgtggtgatggaagtatccagccatgcattgtcattgggcagagttgcgggttcccactttgatgtagctgcgtttaccaacctgtcgcaggatcaccttgatttccaccccaccatggatgattactttgacgcgaaggcattgttcttccgcgcagattctccacttgtggctgacaaacaggtcgtgtgcgtggatgattcttggggtcagcgcatggccagcgtggcagcggatgtgcaaacagtatccacccttgggcaagaagcagacttcagcgctacagacatcaatgtcagcgactctggcgcccagagttttaagatcaacgccccctcaaaccagtcctaccaggtcgagctagctcttccaggtgcgttcaacgttgctaacgccacgttggcatttgccgctgcggcacgcgtgggtgttgatggcgaagcgtttgctcgaggcatgtccaaggtcgcggttccaggccgtatggaacgcattgatgagggacaagacttccttgcagtggtggattatgcccacaagcctgctgcagtggctgctgtgttggatacgttgaggacccagattgacgggcgcctcggagtggttatcggtgctggtggagaccgcgattccaccaagcgtggccccatggggcagttgtccgcacagcgtgctgatctagttattgtcactgatgacaaccctcgttcagaggtgcctgccacgattcgcgcagcagtcactgcaggagcacagcagggtgcttcagagtccgaacgaccggtggaagtcctagaaattggtgaccgtgcagaagcaattcgcgttttggtcgagtgggcacagcctggagatggcattgtagtagctggaaaaggccatgaagttggacaactagttgctggtgtcacccaccattttgatgaccgcgaagaagttcgcgctgctttgacagaaaagctcaacaataaacttccccttactacggaagaaggatag

SEQ ID NO: 44 - DNA, 1566 bp, Corynebacterium glutamicum
atggcaaccacgttgctggacctcaccaaacttatcgatggcatcctcaagggctctgcccagggcgttcccgctcacgcagtaggggaacaagcaatcgcggctattggtcttgactcctccagcttacctacctcggacgctatttttgctgcagttccaggaacccgcactcacggcgcacagtttgcaggtacggataacgctgcgaaagctgtggccattttgactgacgcagctcgccttgaggtgctcaacgaagcaggagagacccgcccagtcatcgttgttgatgatgtccgcgcagtacttggcgcagcatcatcaagcatttatggcgatccttcaaaagatttcacgctcattggagtcactggaacctcaggtaaaaccaccaccagctacctcttggaaaaaggactcatggaggcaggccacaaagttggtttgatcggcaccacaggtacacgtattgacggggaagaagtacccacaaagctcaccactccagaagcgccgactctgcaggcattgtttgctcgaatgcgcgatcacggtgtcacccacgtggtgatggaagtatccagccatgcattgtcattgggcagagttgcgggttcccactttgatgtagctgcgtttaccaacctgtcgcaggatcaccttgatttccaccccaccatggatgattactttgacgcgaaggcattgttcttccgcgcagattctccacttgtggctgacaaacaggtcgtgtgcgtggatgattcttggggtcagcgcatggccagcgtggcagcggatgtgcaaacagtatccacccttgggcaagaagcagacttcagcgctacagacatcaatgtcagcgactctggcgcccagagttttaagatcaacgccccctcaaaccagtcctaccaggtcgagctagctcttccaggtgcgttcaacgttgctaacgccacgttggcatttgccgctgcggcacgcgtgggtgttgatggcgaagcgtttgctcgaggcatgtccaaggtcgcggttccaggccgtatggaacgcattgatgagggacaagacttccttgcagtggtggattatgcccacaagcctgctgcagtggctgctgtgttggatacgttgaggacccagattgacgggcgcctcggagtggttatcggtgctggtggagaccgcgattccaccaagcgtggccccatggggcagttgtccgcacagcgtgctgatctagttattgtcactgatgacaaccctcgttcagaggtgcctgccacgattcgcgcagcagtcactgcaggagcacagcagggtgcttcagagtccgaacgaccggtggaagtcctagaaattggtgaccgtgcagaagcaattcgcgttttggtcgagtgggcacagcctggagatggcattgtagtagctggaaaaggccatgaagttggacaactagttgctggtgtcacccaccattttgatgaccgcgaagaagttcgcgctgctttgacagaaaagctcaacaataaacttccccttactacggaagaaggatag

SEQ ID NO: 45 - DNA, 34 bp, artificial
  oligonucleotide
aaggagatatagatatgagtactgcactcgcaac

SEQ ID NO: 46 - DNA, 21 bp, artificial
  oligonucleotide
tcatgctgccaccttctgctc

SEQ ID NO: 47 - DNA, 22 bp, artificial
  oligonucleotide
gtaactattgccgatgataagc

SEQ ID NO: 48 - DNA, 24 bp, artificial
  oligonucleotide
cggcgtttcacttctgagttcggc

SEQ ID NO: 49 - DNA, 35 bp, artificial
  oligonucleotide
aaggagatatagatatgactaagcaaccaccaatc

SEQ ID NO: 50 - DNA, 22 bp, artificial
  oligonucleotide
cggttattcctctgaattatcg

SEQ ID NO: 51 - DNA, 44 bp, artificial
  oligonucleotide
gcagatctaaggagatatacatatgactaagcaaccaccaatcg

SEQ ID NO: 52 - DNA, 28 bp, artificial
  oligonucleotide
gcgcgaattccaggctgaattattcctc

SEQ ID NO: 53 - DNA, 22 bp, artificial
  oligonucleotide
cgccgacatcataacggttctg

SEQ ID NO: 54 - DNA, 22 bp, artificial
  oligonucleotide
ttatcagaccgcttctgcgttc

SEQ ID NO: 55 - DNA, 44 bp, artificial
  oligonucleotide
cggcagatctaaggagatatacatatgagtactgcactcgcaac

SEQ ID NO: 56 - DNA, 28 bp, artificial
  oligonucleotide
gcgcggaattcatgctgccaccttctgc

SEQ ID NO: 57 - DNA, 25 bp, artificial
  oligonucleotide
cgagtactacaaacgcggccataac

SEQ ID NO: 58 - DNA, 36 bp, artificial
  oligonucleotide
gtcggaagaggcatagaattccgtcagccagtttag

SEQ ID NO: 59 - DNA, 32 bp, artificial
  oligonucleotide
gctgacggaattctatgcctcttccgaccatc

SEQ ID NO: 60 - DNA, 25 bp, artificial
  oligonucleotide
atacaattgaacaaagccgccgtcc

SEQ ID NO: 61 - DNA, 23 bp, artificial
  oligonucleotide
cattggtcacctttggcgtgtgg

SEQ ID NO: 62 - DNA, 24 bp, artificial
  oligonucleotide
aatcaatgagcgccgtgaagaagg

SEQ ID NO: 63 - DNA, 100 bp, artificial
  oligonucleotide
atgcatcatcaggagtacggataaaatgcttgatggtcggaagaggcataaattccgtcagccagtttagtctgaccatctcatctgtaacatcattggc

(19) United States
(12) Patent Application Publication
(10) Pub. No.: US 2016/0298094 A1
(43) Pub. Date: Oct. 13, 2016

(54) METHOD OF IDENTIFYING A CELL WITH AN INTRACELLULAR CONCENTRATION OF A SPECIFIC METABOLITE, WHICH INTRACELLULAR CONCENTRATION IS INCREASED IN COMPARISON WITH THE CELL'S WILDTYPE, WHERE THE MODIFICATION OF THE CELL IS ACHIEVED BY RECOMBINEERING

(71) Applicant: FORSCHUNGSZENTRUM JÜLICH GMBH, Jülich (DE)

(72) Inventors: Stephan BINDER, Eschweiler (DE); Lothar EGGELING, Juelich (DE); Michael BOTT, Juelich (DE)

(21) Appl. No.: 14/651,502

(22) PCT Filed: Nov. 15, 2013
(86) PCT No.: PCT/DE2013/000683

§ 371 Date: Jun. 11, 2015

(30) Foreign Application Priority Data
Dec. 14, 2012 (DE) 10 2012 024 435.5

(51) Int. Cl. C12N 9/12; C12Q 1/68; C12Q 1/48; C12Q 1/04

(52) U.S. Cl. CPC C12N 9/1241; C12Q 1/04; C12Q 1/689; C12Y 207/07; C12Q 1/485; C12Q 2600/156

(57) ABSTRACT

A method for identifying a cell having an intracellular concentration of a particular metabolite that is increased compared to the wild type of the cell, wherein the modification of the cell is achieved by recombineering, and to a method for producing a production cell that is genetically modified compared to the wild type of the cell and has optimized production of a particular metabolite, to a method for producing this metabolite, and to nucleic acids suited therefor. A gene coding for a recombinase, which is homologous to a known recombinase gene, is transformed in a cell using a vector, and a DNA containing at least one modified gene G1 to Gn, or at least one mutation M1 to Mn, is inserted into the cell, and the cell that has highest metabolite production is identified by way of metabolite sensors. A mutation, which is considered to the cause for the increased production, is isolated from this cell, and the gene or the mutation is removed and inserted into a production strain, which thereby exhibits increased production of the metabolite.

Method for Identifying a Cell having an Intracellular Concentration of a Particular Metabolite that is Increased Compared to the Wild Type of the Cell, wherein the Modification of the Cell is Achieved by Recombineering, to a Method for Producing a Production Cell that is Genetically Modified Compared to the Wild Type of the Cell and has Optimized Production of a Particular Metabolite, to a Method for Producing this Metabolite, and to Nucleic Acids Suited Therefor.

The invention relates to a method for identifying a cell having an intracellular concentration of a particular metabolite that is increased compared to the wild type of the cell, wherein the modification of the cell is achieved by recombineering, to a method for producing a production cell that is genetically modified compared to the wild type of the cell and has optimized production of a particular metabolite, to a method for producing this metabolite, and to nucleic acids suited therefor.

Microorganisms have been used on a large scale for decades to produce low molecular weight molecules. For example, low molecular weight molecules are natural bacterial metabolites such as amino acids (EP 1070132 B1, WO 2008/006680 A8), nucleosides and nucleotides (EP 2097512 C1, CA 2297613 C1), fatty acids (WO 2009/071878 C1, WO 2011/064393 C1), vitamins (EP 0668359 C1), organic acids (EP 0450491 B1, EP 0366922 B1) or sugars (EP 0861902 C1, U.S. Pat. No. 3,642,575 A). Low molecular weight molecules produced by bacteria are also molecules that are formed by the expression of heterologous genes stemming from plants, for example. These are plant active agents. These include, for example, taxol (WO 1996/032490 C1, WO 1993/021338 C1), artemisinin (WO 2009/088404 C1), and further molecules belonging to the classes of isoprenoids, phenylpropanoids or alkaloids (Marienhagen J, Bott M, 2012, J Biotechnol., doi.org/10.1016/j.jbiotec.2012.06.001). In addition to molecules, or precursors of molecules of plant origin, it is generally also possible to obtain such molecules by using microorganisms that are of commercial interest. These include, for example, hydroxyisobutyric acid to produce methacrylates (PCTIEP2007/055394), diamines to produce plastics (JP 2009-284905 A), or alcohols for use as fuel (WO 2011/069105 C2, WO 2008/137406 C1).

Gram-negative bacteria, gram-positive bacteria and yeasts are suitable microorganisms for producing low molecular weight molecules. Suitable bacteria are, for example, Escherichia species belonging to the genus Enterobacter, such as Escherichia coli, or Bacillus species belonging to the genus Firmicutes, such as Bacillus subtilis, or Lactococcus species belonging to the genus Firmicutes, such as Lactococcus lactis, or Lactobacillus species such as Lactobacillus casei, Saccharomyces species belonging to the genus Ascomycetes such as Saccharomyces cerevisiae, or Yarrowia species such as Yarrowia lipolytica, or Corynebacterium species belonging to the genus Corynebacterium.

Corynebacterium efficiens (DSM44549), Corynebacterium thermoaminogenes (FERM BP-1539) and Corynebacterium ammoniagenes (ATCC6871) are preferred among the corynebacteria, in particular Corynebacterium glutamicum (ATCC13032). Several species of Corynebacterium glutamicum are also known by different names in the related art. These include, for example, Corynebacterium acetoacidophilum ATCC13870, Corynebacterium lilium DSM20137, Corynebacterium melassecola ATCC 17965, Brevibacterium flavum ATCC14067, Brevibacterium lactofermentum ATCC13869, Brevibacterium divaricatum ATCC14020, and Microbacterium ammoniaphilum ATCC15354.

To achieve the formation and production of low molecular weight molecules, separate genes of the microorganism, or homologous genes or heterologous genes of the synthesis pathways of the low molecular weight molecules are expressed, or the expression thereof is intensified, or the mRNA stability thereof is increased. For this purpose, the genes can be introduced into the cell on plasmids or vectors, or they can be present on episomes or be integrated into the chromosome. It is also possible to increase the expression of the intracellular chromosomally encoded genes. This is achieved by appropriate mutations in the chromosome in the region of the promoter, for example. It is also possible to introduce other mutations resulting in product increases into the chromosome, which influence mRNA stability, for example, or which influence the osmotic stability or the resistance to pH fluctuations, or genes whose function is not known, but which favorably affect product formation. In addition, homologous genes or heterologous genes are inserted into the chromosome, or they are inserted so that they are present in the chromosome in multiple copies.

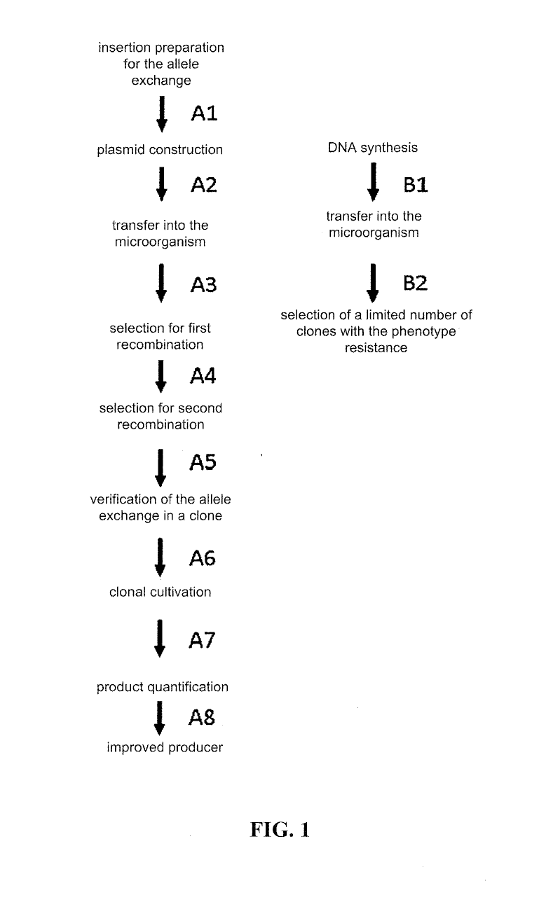

The deliberate insertion of mutations or genes into the genome necessitates the construction of a plasmid, which is produced by in vitro recombination of DNA sequences using restriction endonucleases and DNA ligases. The entire procedure for deliberately introducing chromosomal mutations further comprises the following steps to achieve the in vivo exchange, the test for successful exchange, and finally the test for increased product formation. This requires a plurality of steps, A1 to A8, which are schematically listed in FIG. 1 (on the left). This method is employed for many bacteria used to produce small molecules. Examples include Corynebacterium glutamicum (Small mobilizable multi-purpose cloning vectors derived from the Escherichia coli plasmids pK18 and pK19: selection of defined deletions in the chromosome of Corynebacterium glutamicum. Schäfer A, Tauch A, Jäger W, Kalinowski J, Thierbach G, Pühler A. Gene. 1994 Jul. 22; 145(1):69-73), or Pseudomonas aeruginosa (Allelic exchange in Pseudomonas aeruginosa using novel ColE1-type vectors and a family of cassettes containing a portable oriT and the counter-selectable Bacillus subtilis sacB marker. Schweizer H P. Mol Microbiol. 1992 May; 6(9):1195-204), or Bacillus subtilis (Construction of a modular plasmid family for chromosomal Integration in Bacillus subtilis. Gimpel M, Brantl S. J Microbiol. Methods. 2012 November; 91(2):312-7), or clostridia (Novel system for efficient isolation of clostridium double-crossover allelic exchange mutants enabling markerless chromosomal gene deletions and DNA Integration. Al-Hinai M A, Fast A G, Papoutsakis E T. Appl. Environ Microbiol. 2012 November; 78(22):81 12-21).

The deliberate insertion of mutations or genes into the chromosome necessitates the in vitro recombination of DNA sequences using restriction endonucleases and DNA ligases to produce a plasmid (FIG. 1, A1). The plasmids required for this purpose are plasmids that do not replicate in the desired producer under suitable conditions. After the plasmid has been introduced into the microorganism by electroporation, chemical or ballistic transformation (FIG. 1, A2), the integration into the chromosome is carried out. Subsequent to the integration, a selection is carried out through vector-mediated resistance (FIG. 1, A3). Suitable plasmids are pBRH1 (WO2003076452C2) or pWV01 (U.S. Pat. No. 6,025,190), for example, which are no longer able to replicate in Azetobacter or Bacillus after transformation (FIG. 1, A2) due to an increase in the temperature in the cell, so that the vector is inserted into the chromosome of resistant cells. The plasmid pK19mobsacB is not able to replicate in Corynebacteria, such C. glutamicum, from the outset, so that in the presence of kanamycin only clones are selected, in which the integration of the vector into the chromosome takes place by homologous recombination (Schafer et al., Gene (Genes) 145, 69-73 (1994) (FIG. 1, A3). These non-replicating plasmids serve as vectors for the directed mutation of genes in the chromosome, for mutating promoter sequences, deleting sequences or exchanging sequences, or for inserting new genes into the chromosome. This method is complex since the plasmids must be constructed individually in vitro. It is also complex because initially the insertion of the plasmid, together with the sequences that are to be exchanged, into the chromosome is carried out using appropriate selection methods, such as selection for antibiotic resistance or the described temperature increase, and the loss of the plasmid from the chromosome is achieved in a subsequent step (FIG. 1, A4). It is only through subsequent tests, which are typically PCR amplifications, that it is possible to check whether the sequences that are to be exchanged in fact remain in the chromosome as desired (FIG. 1, A5).

In this way a single clone is constructed, which thereafter is cultivated (FIG. 1, A6), the product of which is quantified (FIG. 1, A7), and thus optionally an improved producer is obtained (FIG. 1, A8). This technique of plasmid construction and homologous recombination to obtain microbial producers is widely used, for example to achieve allelic exchanges or deletions in C. glutamicum or E. coli (U.S. Pat. No. 8,293,514; U.S. Pat. No. 8,257,943; U.S. Pat. No. 8,216,820; WO 2008/006680 A8; EP 2386650 C1).

Of late, what is known as “recombineering” has been introduced as another method of deliberate genome mutation. Introducing mutations requires far fewer steps than the insertion of mutations by way of plasmids (FIG. 1, right, B1 to B2). Recombineering utilizes phage or prophage genes, bringing about the homologous recombination between the chromosomal DNA and externally supplied DNA. In the simplest case, this DNA is used as commercially synthesized single-stranded DNA. It is also possible to use double-stranded DNA amplified by way of PCR. If suitable phage or prophage genes are present, this method requires only few steps. The drawback, however, is that this method is essentially limited to the introduction of mutations that allow growth on selective medium, such as the introduction of antibiotic resistances, because other mutations cannot be detected. The direct use for fast production of product-forming microorganisms is therefore extremely limited and has so far only been described for E. coli and the product lycopene (Programming cells by multiplex genome engineering and accelerated evolution. Wang H H, Isaacs F J, Carr P A, Sun Z Z, Xu G, Forest C R, Church G M. Nature. 2009; 460(7257):894-8). Due to the dyed lycopene, the product formation was inferred in this particular case based on the colony color. So far, it is not possible to directly detect increased product formation for other organisms and other products, such as amino acids or other organic acids. Another drawback is that recombineering is limited to E. coli and a very limited number of other microorganisms, such as Salmonella entenca, Yersinia pseudotuberculosis, Lactobacillus, Bacillus subtilis, and Mycobacterium.

A further problem is that, so far, no general system exists to identify product-forming microorganisms in large cell populations directly after recombineering and to isolate the same from such cell populations. The method previously employed in recombineering involving the selection on petri dishes is, as mentioned above, limited to very special applications and additionally limited in terms of the number of recombinants that are obtained on petri dishes, which makes the method unsuitable for screening large recombinant libraries.

Recombineering is based on homologous recombination, which is mediated by proteins originating from phages or prophages. Two homologous systems are known for Escherichia coli. The RecE/RecT from the Rac prophage, and the Red operon, consisting of red gamma, red beta and red alpha from the bacteriophage lambda. Both systems allow the exchange of freely selectable DNA segments between two different DNA molecules. The exchange of DNA takes place via two homologous (similar or identical) regions that flank the target fragment and have lengths of 30 to 100 base pairs. So as to introduce chromosomal mutations, the DNA molecule carrying the mutation is commercially synthesized as a single strand (FIG. 1, B1), and inserted into E. coli expressing the Red Beta protein. By virtue of the homology between the introduced DNA molecule and the chromosome, the Red Beta protein mediates the recombination and the exchange of the sequences. In this way, mutations in the galK gene of the chromosome of E. coli were corrected. Since only the intact galK gene allows use of galactose, recombinant clones are selected as colonies on petri dishes by growth (FIG. 1, B2) (Rekombineering: in vivo genetic engineering in E. coli, S. enterica, and beyond. Sawitzke J A, Thomason L C, Costantino N, Bubunenko M, Datta S, Court D L. Methods Enzymol. 2007; 421:171-99). It is likewise possible to insert resistance genes or correct corresponding mutations, so that a selection for growth is again possible after successful recombineering. This can also be carried out with genes that code for resistance against chloramphenicol, hygromycin, streptomycin, ampicillin or spectinomycin (Rekombineering: in vivo genetic engineering in E. coli, S. enterica, and beyond. Sawitzke J A, Thomason L C, Costantino N, Bubunenko M, Datta S, Court D L. Methods Enzymol. 2007; 421:171-99). It is thus also possible to insert other genes having an easily selectable phenotype into the chromosome of E. coli, or to mutate these in the chromosome, by way of recombineering. If no other selection option exists, this may be bypassed by various techniques, such as coselection or colony hybridization (Rekombineering: in vivo genetic engineering in E. coli, S. enterica, and beyond. Sawitzke J A, Thomason L C, Costantino N, Bubunenko M, Datta S, Court D L. Methods Enzymol. 2007; 421:171-99), or PCR analysis of clones; however, this requires additional steps and negates the advantage of fast, targeted mutagenesis of the chromosome by way or recombineering. In addition, it requires extraordinarily high recombination frequency, since otherwise hundreds of clones would have to be tested individually. This is the reason why recombineering of the chromosome without clonal cultivations can generally not be used yet to isolate an improved microbial metabolite producer. A special case is the microbial product lycopene, which results in striking red colonies. For example, 20 chromosomal gene loci were mutated by way of iterative, multiple consecutive recombineering with E. coli and a visual qualitative evaluation of the color intensity of colonies on petri dishes so as to achieve increased lycopene formation (Programming cells by multiplex genome engineering and accelerated evolution. Wang H H, Isaacs F J, Carr P A, Sun Z Z, Xu G, Forest C R, Church G M. Nature. 2009; 460(7257):894-8). The limitation of the use of recombineering for the development of microbial producers is based on the absence of a phenotype, for which the selection could be carried out in the majority of microbially produced low molecular weight molecules.

The prior art with respect to product detection also includes metabolite sensors—also known as nanosensors—which can be used to detect increased product formation in individual bacteria. Such metabolite sensors use transcription factors or RNA aptamers to detect low molecular weight metabolites in bacteria and yeasts. Known transcription factor-based metabolite sensors are pSenLys, pSenArg, pSenSer, pSenOAS and pJC1-lrp-bmF-eyfp (WO2011138006; DPA 102012 016 716.4), for example. The metabolite sensor includes a gene sequence coding for an autofluorescent protein, wherein the expression of the autofluorescent protein is dependent on the intracellular concentration of a particular metabolite. The expression of the gene sequence coding for the autofluorescent protein is controlled as a function of the intracellular concentration of the particular metabolite at the transcription level. Depending on the intracellular concentration of the respective metabolite, more or less mRNA is therefore produced, which can be translated by the ribosomes, forming the autofluorescent protein. The microorganism containing the metabolite sensor can be any arbitrary microorganism. Bacteria, yeasts or enterobacteria, such as Escherichia coli, Corynebacterium glutamicum or Saccharomyces cerevisiae, can be mentioned by way of example.

The use of metabolite sensors for inserting cells having increased product formation is based on the increased production and extracellular accumulation of metabolite with increased formation of a metabolite, and the presence of an increased intracellular concentration of the metabolite compared to the wild type (A high-throughput approach to identify genomic variants of bacterial metabolite producers at the single-cell level. Binder S, Schendzielorz G, Stabler N, Krumbach K, Hoffmann K, Bott M, Eggeling L. Genome Biol. 2012 May 28; 13(5):R40; Engineering microbial biofuel tolerance and export using efflux pumps. Dunlop M J, Dossani Z Y, Szmidt H L, Chu H C, Lee T S, Keasling J D, Hadi M Z, Mukhopadhyay A. Mol Syst Biol. 2011 May 10; 7:487).

Metabolite sensors are described for the detection of mutant libraries of microorganism mutants with increased product formation and for sorting these mutants by way of flow cytometry and automatic sorting devices (WO02011138006; DPA 102012 016 716.4). The mutant library in this case had been produced using chemical undirected mutagenesis of the chromosome or by inserting mutations into a plasmid-encoded gene using a faulty polymerase chain reaction. The present invention does not relate to chemical undirected mutagenesis or mutagenesis by way of a faulty polymerase chain reaction.

The drawback of existing techniques for strain development is that so far no technique is available for deliberately introducing mutations into a cellular gene or the chromosome, while also directly identifying an improved metabolite producer as a single cell in cell suspensions, and isolating it from the cell suspensions, without clonal cultivation (petri dishes) after introduction of the mutation.

It is thus the object of the invention to provide such a method for the accelerated development of microbial producers of smaller molecules and overcome the disadvantages of the state of the art.

This object is achieved according to the invention by a cell according to claim 1, by a method according to the other independent claims 6, 7 and 15, by a recombinase gene according to claim 18, by a recombinase according to claim 19, and by nucleic acids according to claim 20.

Advantageous refinements of the invention will be apparent from the dependent claims.

With the cell, the methods, the recombinase genes, the recombinase, the identified genes G1 to Gn and mutations M1 to Mm it is now possible, in a particularly fast way, to create cells for the increased production of metabolites that allow metabolites to be produced at an increased rate compared to the original cell.

The invention will be described hereafter in its general form.

According to the invention, a cell is provided that is genetically modified compared to the wild type thereof and that contains a gene sequence coding for a recombinase and additionally a gene sequence coding for a metabolite sensor.

The cell is preferably a microorganism, especially a bacterium, in particular of the genus Corynebacterium, Enterobacterium or the genus Escherichia, and particularly preferably Corynebacterium glutamicum or Escherichia coli.

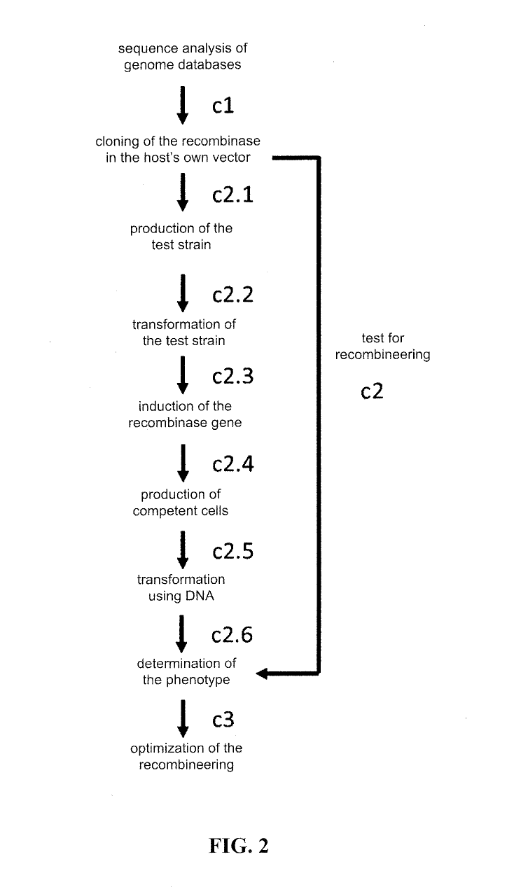

The gene sequence coding for a recombinase can be a sequence that has improved functionality compared to a known recombinase in a desired microorganism. This is a gene sequence coding for a recombinase which codes for a protein that recombines extracellularly added DNA with intracellular DNA. The test for functionality can be carried out as shown schematically in FIG. 2. A gene sequence according to SEQ ID No. 1 or SEQ ID Nos. 7 and 9 has been found to be particularly suitable.

The gene sequence coding for the recombinase can be transformed in the cell and expressed by way of a vector, for example a plasmid, whereby the recombinase is formed.

The recombinase used in the method is characterized by recombining extracellularly added DNA with the intracellular DNA. The recombinase can originate from a larger gene pool, such as metagenome, for example, where possible recombinases are identified by way of sequence comparisons to known recombinases. Such sequence comparisons can also be used to identify possible recombinases in existing databases. Moreover, it is possible to detect proteins that reportedly have recombinase activity, or those suspected to have such activity, as recombinase by way of functional characterization. Recombinases can preferably be isolated from phages or prophages. For example, recombinases can be isolated from prophages of the biotechnologically relevant bacteria Leuconostoc, Clostridia, Thiobacillus, Alcanivorax, Azoarcus, Bacillus, Pseudomonas, Pantoea, Acinetobacter, Shewanella, or Corynebacterium, and the respective related species, and used. Preferred are recombinases homologous to the recombinase RecT of the Rac prophage, or to the recombinase Bet of the Lambda page. The recombinase RecT from the E. coli prophage Rac, the combinase Bet from the E. coli phage Lambda, and the recombinase rCau (Cauri_1962) from Corynebacterium aurimucosum are particularly preferred.

The used gene sequence coding for the metabolite sensor is the sequence of vectors, for example plasmids, coding for proteins that detect metabolites, such as amino acids, organic acids, fatty acids, vitamins or plant active agents and render these visible through fluorescence. The stronger the fluorescence, the higher is the intracellular metabolite concentration. In this way, it is possible to identify a cell having increased fluorescence compared to the genetically unmodified form, and thus increased product formation.

The cell thus modified is suitable for inserting externally supplied DNA molecules that carry the mutations M1 to Mm, or the mutated genes G1 to Gn, into the intracellular DNA, and for indicating increased production of a particular metabolite mediated by the insertion of the DNA by way of fluorescence. The metabolite sensor is selected so as to respond to the detection of the metabolite that is to be formed at an increased rate.

The invention further includes a method for identifying a cell having an intracellular concentration of a particular metabolite that is increased compared to the wild type of the cell, in a cell suspension, comprising the following method steps:

i) providing a cell suspension containing cells of the above-described type;
ii) genetically modifying the cells by recombineering while adding DNA that contains at least one modified gene G1 to Gn, or at least one mutation M1 to Mm, obtaining a cell suspension in which the cells differ in terms of the intracellular concentration of a particular metabolite; and
iii) identifying individual cells in the cell suspension having an increased intracellular concentration of a particular metabolite by fluorescence detection using a metabolite sensor.

The cells used are preferably microorganisms, especially bacteria, in particular of the genus Corynebacterium, Enterobacterium or the genus Escherichia, and particularly preferably Corynebacterium glutamicum, or Escherichia coli.

Recombineering involves methods that are known from the prior art and the methods disclosed in the specific description section, by way of example and without limitation. The recombinase gene is preferably inserted into the cell in a plasmid. It is particularly preferred when a gene according to SEQ ID No. 1 is inserted into the cell for a recombinase.

For this purpose, the cells are preferably transformed using vectors, particularly preferably plasmids, according to the sequences with SEQ ID No. 3 to No. 9.

The metabolites occurring in increased intracellular concentration compared to the wild type can be amino acids, organic acids, fatty acids, vitamins or plant active agents, for example. These are desired products, the production of which is to be improved.

The cell suspension can be cells that are present in a saline aqueous solution, for example, and can optionally contain nutrients.

The DNA used for genetically modifying the cell by recombineering can be single-stranded or double-stranded DNA, or synthetic DNA, or DNA isolated from cells. The DNA can comprise 50 bp to 3 Mb, and DNA having a length of 50 to 150 bp is preferred. The DNA can code for proteins, or parts of proteins, of the producer that is to be genetically modified. It is also possible to use DNA that is homologous to promoter regions, or regions having unknown functions, of the producer that is to be genetically modified. Moreover, the DNA can code for genes or regulatory elements from other organisms than those of the producer to be genetically modified.

In addition to defined DNA molecules, it is also possible to use mixtures of DNA molecules, which is advantageous for creating large genetic diversity, for example.

The insertion may be made into the chromosome or into a plasmid.

Fluorescence detection methods by way of a metabolite sensor are known to a person skilled in the art.

In one embodiment, the invention also relates to a method for producing a production cell that is genetically modified compared to the wild type thereof and has optimized production of a particular metabolite, comprising the following steps:

i) providing a cell suspension containing cells of the above-described type;
ii) genetically modifying the cells by recombineering while adding DNA that contains at least one modified gene G1 to Gn, or at least one mutation M1 to Mm. Obtaining a cell suspension in which the cells differ in terms of the intracellular concentration of a particular metabolite;
iii) identifying individual cells in the cell suspension having an increased intracellular concentration of a particular metabolite by fluorescence detection using a metabolite sensor;
iv) separating the identified cells from the cell suspension;
v) identifying those genetically modified genes G1 to Gn, or those mutations M1 to Mm, in the identified and separated cells that are responsible for the increased intracellular concentration of the particular metabolite; and
vi) produing a production cell that is genetically modified compared to the wild type thereof and has optimized production of the particular metabolite, the genome of the cell comprising at least one of the genes G1 to Gn and/or at least one mutation M1 to Mm.

The same interrelationships that apply to the method for identifying a cell having an increased intracellular concentration of a particular metabolite compared to the wild type of the cell in a cell suspension also apply to the cells, the recombineering, the metabolites, the cell suspension, the methods of fluorescence detection, the vectors, and the DNA inserted into the cells from steps i), ii) and iii).

The separation of the identified cell can be carried out using known methods.

To produce a production cell that is modified compared to the wild type, the cells that were used to identify the increased production and indicate an increased production of metabolites by way of increased fluorescence are isolated.

In these cells, the mutation M1 to Mm and/or in the gene G1 to Gn, or the mutations M1 to Mm are identified in the genes G1 to Gn. This may be done by way of PCR amplification of the target genes in the genes G1 to Gn and/or the mutation types M1 to Mm, with subsequent sequencing. Likewise, sequencing of the genome can be carried out.

The identified product-increasing mutations M1 to Mm and/or genes G1 to Gn are subsequently transferred into the production cell. This may be done by methods that are known to the person skilled in the art from the prior art.

The designation-G1 to Gn is directed to at least one of the genes G1, G2, G3 to Gn that was added to the cell as part of the recombineering and is now considered to be the cause for a particularly good increase in the production of the metabolite.

The designation M1 to Mm-is directed to mutations M1, M2, M3 to Mm that are contained in the genes G1 to Gn and added to the cell in method step ii) and that is now considered to be the cause of a particularly good increase in the production of the metabolite.

These genes or these mutations are isolated from the cell and inserted into the genome of the production cell using known methods. The gene or the mutation, or the genes or the mutations, can be inserted into the chromosomal DNA or a plasmid of the production cell.

These genes or mutations are DNA segments that preferably code for proteins of the steps of a biosynthesis pathway of the desired metabolite, or optionally a metabolic process related thereto. This can also be DNA that is used to favorably influence the promoter activity of genes or the stability of mRNA of genes for product formation.

In particular, the genes according to sequences SEQ ID No. 33 to SEQ ID No. 44 were found, which are suitable for increasing the production of L-lysine.

The invention further relates to a method for producing metabolites, comprising the following method steps:

a) producing a production cell that is genetically modified compared to the wild type thereof and has optimized production of a particular metabolite using a method of the type described above; and
b) cultivating the cell in a culture medium containing nutrients under conditions in which the production cell produces the particular metabolite from the nutrients.

The metabolite thus produced is secreted into the culture medium and can be isolated from the culture medium.

The culture medium or fermentation medium to be used must satisfy the needs of the respective strains in a suitable manner. Suitable culture media are known to the person skilled in the art. Descriptions of culture media for different microorganisms can be found in the handbook &ldquor; Manual of Methods for General Bacteriology” of the American Society for Bacteriology (Washington D.C., USA, 1981). The terms culture medium and fermentation medium, or medium, are mutually interchangeable.

The carbon source used can be sugar and carbohydrates such as glucose, sucrose, lactose, fructose, maltose, molasses, sucrose-containing solutions from sugar beet or sugar cane processing, starch, starch hydrolysate and cellulose, oils and fats such as soy bean oil, sunflower oil, peanut oil and coconut fat, fatty acids such as palmitic acid, stearic acid and linoleic acid, alcohols such as glycerol, methanol and ethanol, and organic acids such as acetic acid or lactic acid.

The nitrogen source used can be organic nitrogen-containing compounds such as peptones, yeast extract, meat extract, malt extract, corn steep liquor, soybean meal and urea, or organic compounds such as ammonium sulfate, ammonium chloride, ammonium phosphate, ammonium carbonate and ammonium nitrate. The nitrogen sources can be used individually or as mixtures.

The phosphorus source used can be phosphoric acid, potassium dihydrogen phosphate or dipotassium hydrogen phosphate, or the corresponding sodium-containing salts.

The culture medium must additionally include salts, for example in the form of chlorides or sulfates or metals, such as sodium, potassium, magnesium, calcium and iron, for example magnesium sulfate or iron sulfate, which are necessary for growth. Finally, essential growth-promoting substances such as amino acids, for example homoserine, and vitamins, for example thiamine, biotin or pantothenic acid, can be used in addition to the above-mentioned substances.

The described charged substances can be added to the culture in the form of a single batch, or fed in an appropriate manner during cultivation.

Alkaline compounds such as sodium hydroxide, potassium hydroxide, ammonia or ammonia water, or acid compounds such as phosphoric acid or sulfuric acid can be used in a suitable manner to control the pH value of the culture. The pH value is generally set to a value of 6.0 to 8.5, and preferably 6.5 to 8. To control foam development, it is possible to use anti-foaming agents, such as fatty acid polyglycol ester. So as to maintain the stability of plasmids, it is possible to add appropriate, selective acting substances, such as antibiotics, to the medium. The fermentation is preferably carried out under aerobic conditions. Oxygen or oxygen-containing gas mixtures, such as air, are added to the culture to maintain these conditions. It is likewise possible to use liquids that are enriched with hydrogen peroxide. The fermentation is optionally carried out at positive pressure, for example at a positive pressure of 0.03 to 0.2 MPa. The temperature of the culture is normally 20° C. to 45° C., preferably 25° C. to 40° C., and particularly preferably 30° C. to 37° C. In batch processes, cultivation preferably continues until a sufficient amount for the measure of obtaining the desired metabolite, such as an amino acid, organic acid, a vitamin or a plant active agent, has formed. This goal is normally reached within 10 to 160 hours. Longer cultivation times are possible with continuous processes. The activity of the microorganisms results in an enrichment (accumulation) of the metabolite in the fermentation medium and/or in the cells of the microorganisms.

Examples of suitable fermentation media can be found in the patent specifications U.S. Pat. No. 5,770,409, U.S. Pat. No. 5,990,350, U.S. Pat. No. 5,275,940, WO 2007/012078, U.S. Pat. No. 5,827,698, WO 2009/043803, U.S. Pat. No. 5,756,345 or U.S. Pat. No. 7,138,266, among others.

The method according to the invention for producing metabolites can be used to particularly effectively produce amino acids, organic acids, vitamins, carbohydrates or plant active agents, for example.

This method is preferably used to produce L-amino acids, nucleotides and plant active agents, and particularly preferably L-lysine.

The invention also relates to a recombinase gene according to SEQ ID no. 1 and the alleles thereof, displaying homology of at least 70%, preferably 80%, particularly preferably 85% and/or 90%, and most preferably 91%, 92%, 93%, 94%, 95%, 96%, 97%, 98% or 99%.

The invention also relates to a recombinase according to SEQ ID no. 2 and the homologous proteins thereof, displaying homology of 95%, 96%, 97%, and preferably of 98% or 99%.

Moreover, nucleic acids according to the sequences of SEQ ID no. 33 to SEQ ID no. 44 form part of the invention, which code for genes that allow particularly productive production strains to be obtained and originate from the cell for the identification of mutations.

The invention will be described hereafter in the specific description section, in more detail but without limitation.

A method, in which a recombinase is identified and used, helps to achieve the object. So as to identify recombinases for biotechnologically relevant bacteria such as Leuconostoc, Clostridia, Thiobacillus, Alcanivorax, Azoarcus, Bacillus, Pseudomonas, Pantoea. Acinetobacter, Shewaniella, and Corynebacterium species, and more particularly Corynebacterium glutamicum, genome databases are analyzed, according to known methods, for proteins which are homologous to known recombinases and which are expected to provide, or which are hoped will provide, an improved function in the desired organism, over that of the known recombinases. Genome databases are readily accessible, for example the database of the European Molecular Biologies Laboratories (EMBL, Heidelberg, Germany and Cambridge, UK), the database of the National Center for Biotechnology Information (NCBI, Bethesda, Md., USA), the database of the Swiss Institute of Bioinformatics (Swissprot, Geneva, Switzerland), or the Protein Information Resource Database (PIR, Washington, D.C., USA), and the DNA Data Bank of Japan (DDBJ, 111 1 Yata, Mishima, 411-8540, Japan).

The aforementioned databases are used to search for proteins that are homologous to known recombinases (FIG. 2, c1), such as RecT from the Rac prophage—(Genetic and molecular analyses of the C-terminal region of the recE gene from the Rac prophage of Escherichia coli K-12 reveal the recT gene. Clark, A. J., Sharma, V., Brenowitz, S., Chu, C. C., Sandler, S., Satin, L, Templin, A., Berger, I., Cohen, A. J. Bacteriol. (1993)), Beta from the Lambda phage (Hendrix, R. W. (1999). All the world's a phage. Proc Nat Acad Sc USA 96: 2192-2197), gp61 from mycobacteriophage Che9c, or gp43 from mycobacteriophage Halo (Rekombineering [sic] mycobacteria and their phages. van Kessel J C, Marinelli L J, Hatfull G F. Nat Rev Microbiol. 2008 November; 6(11):851-857). The search for the homologous proteins is carried out using known algorithms and sequence analysis programs according to known methods that are publicly accessible, for example as described in Staden (Nucleic Acids Research 14, 217-232 (1986)), or Marck (Nucleic Acids Research 16, 1829-1836 (1988)) or by using the GCG program from Butler (Methods of Biochemical Analysis 39, 74-97 (1998)).

According to the invention, the sequences according to the invention also comprise those sequences that display homology (at the amino acid level) or identity (at the nucleic acid level, exclusive of the natural degeneration) of greater than 70%, preferably 80%, more preferably 85% (based on the nucleic acid sequence) or 90% (also based on the polypeptides), preferably greater than 91%, 92%, 93% or 94%, more preferably greater than 95% or 96%, and particularly preferably greater than 97%, 98% or 99% (based on both types of sequences) to one of these sequences, as long as the mode of action or function and purpose of such a sequence are preserved. The term “homology” (or identity) as used herein can be defined by the equation H (%)=[1−V/X]×100, where H denotes homology, X is the total number of nucleobases/amino acids of the comparison sequence, and V is the number of different nucleobases/amino acids of the sequence to be examined based on the comparison sequence. In any case, the term ‘nucleic acid sequences’ coding for polypeptides encompasses all sequences that appear possible according to the proviso of degeneration of the genetic code.

The identity, in percent, to the amino acid sequences indicated in the sequence listing can be readily ascertained by a person skilled in the art using methods known in the prior art. A suitable program that can be used according to the invention is BLASTP (Altschul et al., 1997. Gapped BLAST and PSI-BLAST: a new generation of protein database search programs. Nucleic Acids Res. 25(17): 3389-3402).

According to the invention, the sequences indicated in the sequence listing also comprise nucleic acid sequences hybridized with those listed. A person skilled in the art can find instructions on hybridization, among other things, in “The DIG System Users Guide for Filter Hybridization” from Boehringer Mannheim GmbH (Mannheim, Germany, 1993) and in Liebl et al. (International Journal of Systematic Bacteriology 41: 255-260 (1991)). The hybridization takes place under stringent conditions, which is to say only hybrids are formed, in which probes, for example the nucleotide sequence complementary to the gene, and the target sequence, which is to say the polynucleotides treated with the probe, are at least 70% identical. It is known that the stringency of the hybridization process, including the washing steps, is influenced or determined by varying the buffer composition, the temperature and the salt concentration. The hybridization reaction is generally carried out at relatively low stringency in comparison with the washing steps (Hybaid Hybridisation Guide, Hybaid Limited, Teddington, U K, 1996). For example, a buffer corresponding to 5×SCC buffer at a temperature of approximately 50° C. to 68° C. can be used for the hybridization reaction. Probes can also hybridize with polynucleotides having an identity lower than 70% with the sequence of the probe. Such hybrids are less stable and are removed by washing under stringent conditions. This can be achieved, for example, by lowering the salt concentration to 2×SCC, and optionally subsequently 0.5×SCC (The DIG System User's Guide for Filter Hybridisation, Boehringer Mannheim, Mannheim, Germany, 1995), wherein the temperature is set to approximately 50° C. to 68° C., approximately 52° C. to 68° C., approximately 54° C. to 68° C., approximately 56° C. to 68° C., approximately 58° C. to 68° C., approximately 60° C. to 68° C., approximately 62° C. to 68° C., approximately 64° C. to 68° C., or approximately 66° C. to 68° C. The washing steps are preferably carried out at temperatures of approximately 62° C. to 68° C., preferably 64° C. to 68° C., or approximately 66° C. to 68° C., and particularly preferably 66° C. to 68° C. Optionally, it is possible to lower the salt concentration to a concentration corresponding to 0.2×SCC or 0.1×SSC. By incrementally increasing the hybridization temperature in steps of approximately 1 to 2° C. from 50° C. to 68° C., it is possible to isolate polynucleotide fragments coding for amino acid sequences, which have, for example, at least 70%, or at least 80%, or at least 90% to 95%, or at least 96% to 98%, or at least 99% identity with the sequence of the probe that is used. Further hybridization instructions are available on the market in the form of so-called kits (such as DIG Easy Hyb from Roche Diagnostics GmbH, Mannheim, Germany, Catalog No. 1603558).

The new DNA sequence from Corynebacterium aurimucosum thus determined, which includes the recombinase gene recT (SEQ ID No. 1) and codes for the functional recombinase rCau (SEQ ID No. 2), forms part of the present invention.

Identified recombinases are cloned in a vector, for example a plasmid, that allows the inducible expression of the recombinase gene in the host in which the recombination is carried out (FIG. 2, c2.1). Expression vectors are the state of the art. For example, the vector pCB42 can be used for Leuconostoc or Lactobacillus (Construction of theta-type shuttle vector for Leuconostoc and other lactic acid bacteria using pCB42 isolated from kimchi. Eom H J, Moon J S, Cho S K, Kim J H, Han N S. Plasmid. 2012 January; 67(1):35-43), ptHydA can be used for Clostridia (Girbal L, von Abendroth G, Winkler M, Benton P M, Meynial-Salles I, Croux C, Peters J W, Happe T, Soucaille P (2005) Homologous and heterologous over-expression in Clostridium acetobutylicum and characterization of purified clostridial and algal Fe-only hydrogenases with high specific activities. Appl. Environ Microbiol. 71: 2777-2781), pTF-FC2 can be used for Thiobacillus (Plasmid evolution and interaction between the plasmid addiction stability systems of two related broad-host-range IncQ-like plasmids. Deane S M, Rawlings D E. J Bacteriol. 2004 April; 186(7):2123-33.), x pRED for Alcanivorax (Appl Microbiol Biotechnol. 2006 July; 71(4):455-62. Functional expression system for cytochrome P450 genes using the reductase domain of self-sufficient P450RhF from Rhodococcus sp. NCIMB 9784. Nodate M, Kubota M, Misawa N.), pMG for Bacillus (Construction of a modular plasmid family for chromosomal integration in Bacillus subtilis. Gimpel M, Brantl S. J Microbiol Methods. 2012; 91(2):312-7), pWWO for Pseudomonas (Increasing Signal Specificity of the TOL Network of Pseudomonas putida mt-2 by Rewiring the Connectivity of the Master Regulator XylR. de Las Heras A, Fraile S, de Lorenzo V. PLoS Genet. 2012 October; 8(10):e1002963), pAGA for Pantoea (Characterization of a small cryptic plasmid from endophytic Pantoea agglomerans and its use in the construction of an expression vector. de Lima Procópio RE, Araijo W L, Andreote F D, Azevedo J L. Genet Mol Biol. 2011 January; 34(1): 103-9), pRIO-5 for Acinetobacter (Complete sequence of broad-host-range plasmid pRIO-5 harboring the extended-spectrum-β-lactamase gene blaBES. Bonnin R A, Poirel L, Sampaio J L, Nordmann P. Antimicrob Agents Chemother. 2012 February; 56(2):1116-9), pBBR1-MCS for Shewaniella (Shewanella oneidensis: a new and efficient system for expression and maturation of heterologous [Fe—Fe] hydrogenase from Chlamydomonas reinhardtii. Sybirna K, Antoine T, Lindberg P, Fourmond V, Rousset M, Méjean V, Bottin H. BMC Biotechnol. 2008 Sep. 18; 8:73), or pZ1 (Menkel et al., Applied and Environmental Microbiology (1989) 64: 549-554) or pCL-TON for Corynebacterium species, in particular C. glutamicum (A tetracycline inducible expression vector for Corynebacterium glutamicum allowing tightly regulable gene expression. Lausberg F, Chattopadhyay A R, Heyer A, Eggeling L, Freudl R. Plasmid. 2012 68(2): 142-7). An overview article on expression plasmids in Corynebacterium glutamicum is described by Tauch et al. (Journal of Biotechnology 104, 27-40 (2003)).

According to a preferred embodiment of the vectors according to the invention, the vectors are pCLTON2-bet (SEQ ID No. 3), pCLTON2-recT (SEQ ID No. 4), pCL-TON2-gp43 (SEQ ID No. 5), pCLTON2-gp61 (SEQ ID No. 6), pCLTON2-rCau (SEQ ID No. 7), pEKEx3-recT (SEQ ID No. 8), and pEKEx3-bet (SEQ ID No. 9).

The vectors thus produced are tested for activity of the recombinase in the respective host (FIG. 2, c2). The activity test includes the production of a test strain of the host in which an easy-to-test phenotype is to be produced by way of recombineering (FIG. 2, c2.1). The further steps include transforming the test strain (FIG. 2, c2.2), inducing the expression of the recombinase gene (FIG. 2, c2.3), producing competent cells to receive linear DNA (FIG. 2, c2.4), transforming the competent cells using linear DNA (FIG. 2, c2.5), and testing for the production of the phenotype (FIG. 2, c2.6). If the expected phenotype can be produced, recombineering has taken place. The individual steps, c2.1 to c2.6, are known to the person skilled in the art. For example, a defective antibiotic resistance gene is inserted into the chromosome of the test strain as an easy-to-test phenotype (FIG. 2, c2.1), the function of which is restored by successful recombineering. Genes that impart resistance against kanamycin, chloramphenicol, hygromycin, streptomycin, ampicillin or spectinomycin, are available as antibiotic resistance genes. It is also possible to use genes that allow growth on a particular substrate as selection marker, such as the galK gene coding for galactokinase. The transformation of the test strain using the test plasmid expressing the recombinase (FIG. 2, c2.2) is carried out according to known methods, for example electroporation, chemical transformation or ballistic transformation. So as to induce the recombinase gene (FIG. 2, c2.3) in the expression vector, the inductor specified by the vector is added to the medium. This procedure is known to the person skilled in the art, and the inductor is for example, isopropyl-β-D-thiogalactopyranoside, anhydrotetracycline, sakacin or acetamide. The further steps, such as producing competent cells (FIG. 2, c2.4), transforming the cells (FIG. 2, c2.5), and testing for the production of the phenotype by plating out on petri dishes (FIG. 2, c2.6), are standard microbiological methods and likewise known to the person skilled in the art.

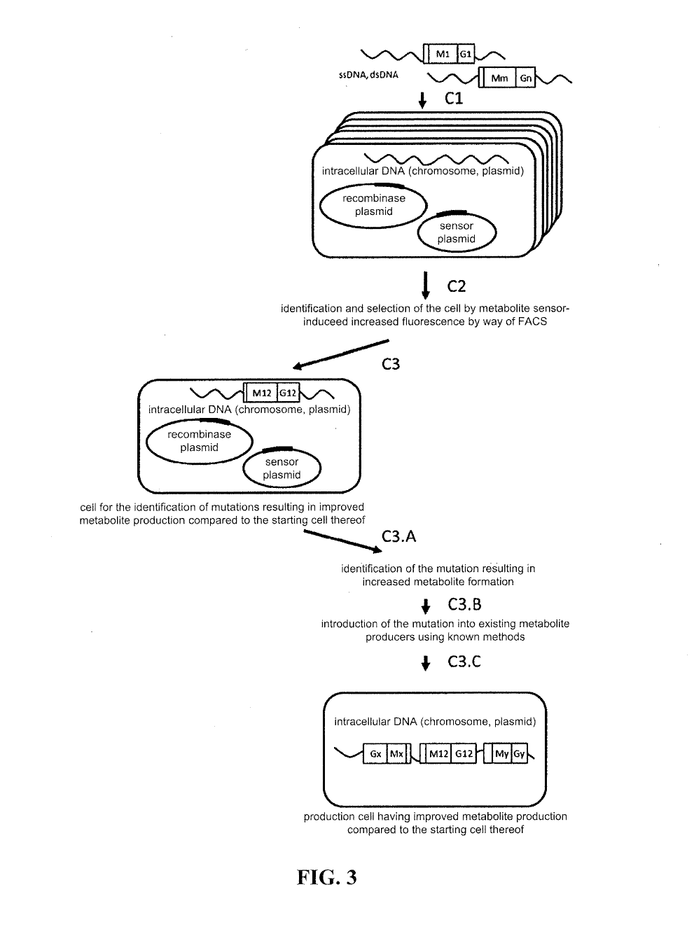

If the desired phenotype is produced according to the described method, thereafter the recombineering process is preferably optimized (FIG. 3, c3). This includes varying the induction time in the range from thirty minutes to six hours, varying the DNA used for recombineering, and varying the regeneration and segregation time, and optionally further parameters that are known to the person skilled in the art.

The DNA used for recombineering is single-stranded DANN, which is synthesized by commercial providers and can be up to 300 base pairs long. The desired mutation to be introduced into the chromosome is present at the center of the DNA and, flanking the same, the DNA includes sequences that are homologous to the chromosomal sequence of the host (U.S. Pat. No. 7,144,734). The optimization includes the test of DNA of varying lengths. The DNA used is DNA having a length of 20 to 300 base pairs, and preferably of 100 base pairs. The optimization includes the test of DNA of varying quantities, wherein 0.2 to 30 micrograms is used for transformation, and preferably 10 micrograms. The optimization further includes the test of DANN that is either homologous to the sense strand or to the antisense strand, wherein preferably the DNA that is homologous to the complementary strand is used (U.S. Pat. No. 7,674,621). The individual optimization steps are known to the person skilled in the art and, for example, are described for E. coli (Rekombineering: in vivo genetic engineering in E. coli, S. enterica, and beyond. Sawitzke J A, Thomason L C, Costantino N, Bubunenko M, Datta S, Court D L. Methods Enzymol. 2007; 421:171-99), Bacillus subtilis (Bacillus subtilis genome editing using ssDNA with short homology regions. Wang Y, Weng J, Waseem R, Yin X, Zhang R, Shen Q. Nucleic Acids Res. 2012 July; 40(12):e91), or Lactococcus (High efficiency Rekombineering in lactic acid bacteria. van Pijkeren J P, Britton R A. Nucleic Acids Res. 2012, 40(10):e76).

To carry out the recombineering so as to obtain a microbial producer (FIG. 2, C1-C4), the chromosomal gene locus to be mutated is selected. These can be known genes, genes having unknown functions, or intergenic regions. The producers are, for example, genes or promoter regions of genes involved in anabolism or catabolism, or in regulatory processes, or those that influence the half life of mRNA or proteins.

The DNA used for recombination is synthesized or produced by way of PCR amplification. It is 30 to 3000 base pairs long and has the organizational structure A-B-C. B is the desired mutation located at the center. In the case of an insertion, this may be a sequence of 1 to 3000 base pairs, preferably one of 1 to 1000, more preferably one of 1 to 100, and particularly preferably one of 1 base pair. The sequences A and C are homologous to chromosomal sequences. In the synthesized DNA, they are in each case 20 to 100 base pairs long. In the case of a deletion desired in the chromosome, B is zero base pairs long, and A and C are homologous to sequences in the chromosome that directly adjoin the region to be deleted. In the synthesized DNA, the sequences A and C are 20 to 100 base pairs long. The deletion in the chromosome can be 1 base pair or up to 10 kb. For exchange of bases in the chromosome, B represents the region to be exchanged, which can comprise 1 to 50 base pairs. The sequences A and C are homologous to chromosomal sequences adjoining the region to be exchanged. In the synthesized DNA, they are 20 to 100 base pairs long. DNA syntheses are carried out, for example, by Genescript (GenScript USA Inc., 860 Centennial Ave., Piscataway, N.J. 08854, USA), or Eurofins (Eurofins MWG Operon, Anzingerstr. 7a, 85560 Ebersberg, Germany), or DNA 2.0 (DNC2.0; DNA 2.0] Headquarters, 1140 O'Brien Drive, Suite A, Menlo Park, Calif. 94025, USA).

The synthesized DNA, or the DNA produced by way of PCR amplification, is inserted by transformation into the microorganism that expresses the recombinase and contains a metabolite sensor (FIG. 3, C1). Microorganisms comprising such metabolite sensors have been described (WO02011138006, DPA 102012 016 716.4, DPA 10 2012 017 026.2). The DNA used for transformation and recombination is a defined DNA sequence, as described above.

However, it is also possible to use defined mixtures of different DNA sequences for transformation and recombination. These mixtures are preferably used during the exchange of bases in the chromosome in the region “B,” where “B” represents the region to be exchanged in the organizational structure A-B-C of the DNA sequence. For example, it is possible to simultaneously exchange various amino acids in one position in the polypeptide in a gene in a population of microorganisms. It is also possible to simultaneously exchange various amino acids in different positions in the polypeptide. It is also possible to simultaneously exchange various nucleotides in a promoter region. The corresponding DNA mixtures can be directly produced by mixing individual defined DNA sequences, or they can already be synthesized by the manufacturer as mixtures, whereby up to several thousand different DNA molecules are present in a batch, which are then also used in a batch for transformation and recombination (FIG. 3, C1). Such DNA mixtures including a wide variety of sequences can be procured commercially. For example, “Combinatorial Libraries” or “Controlled Randomized Libraries” or “Truncated libraries” are offered by Life Technologies GmbH, Frankfurter Straβe 129B, 64293 Darmstadt, which can be used directly for recombineering.

Moreover, it is also possible to used undefined DNA sequences for transformation and recombination. This is genomic DNA from existing producers, for example. In this way, it is possible to identify DNA segments and/or mutations and/or genes that favor product formation.

Subsequent to transforming the recombinase- and nanosensor-containing microorganisms, regeneration is carried out in a complex medium, as is known to the person skilled in the art and described, for example, for E. coli (Hanahan, D. Studies on transformation of Escherichia coli with plasmids. J Mol Biol. 1983; 166(4): 557-80), or Corynebacterium (Tauch A, Kirchner O, Ldffler B, Götker S, Pühler A, Kalinowski J. Efficient electrotransformation of corynebacterium diphtheriae with a mini-replicon derived from the Corynebacterium glutamicum plasmid pGC1. Curr Microbiol. 2002; 45(5):362-7). Following the regeneration, the cells are optionally transferred into a minimal medium for segregation whereupon, in accordance with the method according to the invention, the product analysis is carried out directly in individual cells by way of flow cytometry and selection of the producer (FIG. 3, C2). The selected individual cells are placed on medium in petri dishes, or placed directly into microtiter plates containing liquid medium for further cultivation. Details regarding the analysis of cell suspensions by way of flow cytometry can be found in Sack U, Tarnok A, Rothe G (publisher): Zellulare Diagnostik. Grundlagen, Methoden und klinische Anwendungen der Durchflusszytometne (Cellular Diagnostics. Fundamentals, Methods and Clinical Applications of Flow Cytometry), Basel, Karger, 2007, pages 27-70, for example. Suitable flow cytometers that analyze up to 100,000 cells per second and have a sorting option include, for example, the device Aria-Ill (BD Biosciences, 2350 Qume Drive, San Jose, Calif., USA, 95131, 877.232.8995) or the device MoFlo-XDP (Beckman Coulter GmbH, Europark Fichtenhain B 13, 47807 Krefeld, Germany).

Subsequent to the producer isolation (FIG. 3. C3), the verification of the product formation properties is carried out in shake flasks or microtiter plates. The particularly suited producer is selected. It produces more of the microbially produced product than the starting strain used in step C1 (FIG. 3) for DNA transfer. The product-increasing mutation that has taken place can be identified (FIG. 3, C3.A) by sequencing the genome in the regions that are defined by the DNA added in step C1, or the entire genome, or plasmid-encoded DNA. The corresponding mutations M1 to Mm and/or genes G1 to Gn are optionally transferred in other producer strains using known methods (FIG. 3, C3.B) so as to further improve an existing metabolite producer (FIG. 3, C3.C).

The invention will now be described in more detail based on figures and non-limiting example.

FIG. 1: (on the left) To provide an understanding of the invention, the figure shows an illustration of the flow of the method according to the related art, which is to say the principle of producing a chromosomal mutation using steps A1 to A8, starting from the construction of a specific plasmid (A1), through two selection steps on petri dishes (A3 to A4) and clonal cultivation (A6), to the test for improved production (A7 to A8), and (on the right) the principle of producing a chromosomal mutation by recombineering, starting from synthetic DNA (B1), and the selection of resistant clones on petri dishes or dyed clones on petri dishes (B2).

FIG. 2: The figure shows the development of recombineering according to the invention for a microorganism that is relevant for the biotechnological production of low molecular weight molecules. Sequence analyses are used to identify recombinases (c1), which are inserted into suitable expression vectors (c2.1). Following steps that cause high recombination expression in the host and enable the host to absorb DNA (c2.2 to c2.4), the DNA is added as single-stranded or double-stranded DNA (c.25), and selection for a suitable phenotype of the test strain is carried out (c2.6). In the overall test for recombineering (c2), optimization of the same is subsequently carried out (c3).

FIG. 3: The recombineering according to the invention is combined with cytometric product analysis using metabolite sensors for the isolation of microbial metabolite producers and the further use of mutations thus identified to improve existing metabolite producers. DNA is added to the cells (C1) expressing the recombinase and containing the sensor plasmid including the metabolite sensor. By way of recombinase, the added DNA is inserted into the cells together with the mutated genes G1 to Gn having the mutations M1 to Mm. Cells having increased product formation, and thus increased fluorescence, are isolated using high throughput flow cytometry and selection (FACS) (C2), thus providing a cell for the identification of the mutations resulting in improved metabolite formation (C3). This cell optionally also already represents an improved metabolite producer. Using known methods, the genome or plasmid of the cell resulting from step C3 is sequenced (C3.A) to identify the mutations M1 to Mm in the genes G1 to Gn, so as to insert these into existing metabolite producers (C3.C) to further improve the same, using known methods (C3.B).

### EXAMPLE 1

### Identification of a Recombinase

Using the sequence of RecT from the Rac prophage of Escherichia coli stored under accession number CAD61789.1 in the database of the National Center for Biotechnology Information (NCBI, Bethesda, Md., USA), a homology search was carried out by way of the Blast program, BLAST 2.2.27+(Wheeler, David; Bhagwat, Medha (2007). “Chapter 9, BLAST QuickStart”. In Bergman, Nicholas H. Comparative Genomics Volumes 1 and 2. Methods in Molecular Biology. 395-396. Totowa, N.J.: Humana Press). The homology search was carried out with comparison to all proteins coded in the genomes of the following Corynebacteria species: C. accolens, C. ammoniagenes, C. amycolatum, C. aurimucosum, C. bovis, C. diphtheriae, C. efficiens, C. genitalium, C. glucuronolyticum, C. glutamicum, C. jeikeium, C. kroppenstedtii, C. lipophiloflavum, C. matruchotii, C. nuruki, C. pseudogenitalium, C. pseudotuberculosis, C. resistens, C. striatum, C. tuberculostearicum. C. ulcerans, C. urealyticum, and C. variabile.

The result obtained was the sequence cauri_1962, which codes for a protein having a length of 272 amino acids, of which 41% are identical to, and 61% similar to, the sequence of RecT. The DNA sequence from C. aurimucosum thus determined, which contains the recombinase gene recT, is indicated as SEQ ID No. 1 and the protein sequence is indicated as SEQ ID No. 2.

### EXAMPLE 2

### Cloning Recombinases

Recombinases were cloned in the expression vector pCLTON2 (A tetracycline inducible expression vector for Corynebacterium glutamicum allowing tightly regulable gene expression. Lausberg F, Chattopadhyay A R, Heyer A, Eggeling L, Freudl R. Plasmid. 2012 68(2):142-7), and in the vector pEKEx3 (The E2 domain of OdhA of Corynebacterium glutamicum has succinyltransferase activity dependent on lipoyl residues of the acetyltransferase AceF. Hoffelder M, Raasch K, van Ooyen J, Eggeling L. J Bacteriol. 2010; 192(19):5203-11).

### EXAMPLE 2a

### Production of pCLTON2-Bet

To clone Bet, the vector pSIM8 (Rekombineering: in vivo genetic engineering in E. coli, S. enterica, and beyond. Sawitzke J A, Thomason L C, Costantino N, Bubunenko M, Datta S, Court DL. Methods Enzymol. 2007; 421:171-99) was isolated from E. coli using the QIAGEN Plasmid Plus Maxi Kit (order no. 12963). This plasmid served as a template for PCR amplification using the primer pairs bet-F and bet-R.

bet-F
aaggagatatagatATGAGTACTGCACTCGCAAC
bet-R
TCATGCTGCCACCTTCTGCTC

The resulting fragment of 0.8 kb was isolated by way of gel isolation using the Minielute Extraction Kit (order no. 28704) from Quiagen, filled with the Klenow fragment, and subsequently phosphorylated with T4 polynucleotide kinase from Fermentas (order no. EK0031). The vector pCLTON2 (A tetracycline inducible expression vector for Corynebacterium glutamicum allowing tightly regulable gene expression. Lausberg F, Chattopadhyay A R, Heyer A, Eggeling L, Freudl R. Plasmid. 2012 68(2): 142-7) was cut S times and dephosphorylated using shrimp alkaline phosphatase from Fermentas (order no. EF0511). The fragment and the vector were ligated using the Rapid DNA Ligation Kit from Roche (order no. 11 635 379 001) and used to transform E. coli DH5. Transformed cells were plated out onto 100 ng/ml spectinomycin-containing complex medium.

To test for desired ligation products, a colony PCR was carried out using the primer pairs PcI_fw and Pcl_rv-pEKEx2_fw.

Pcl_fw
GTAACTATTGCCGATGATAAGC
Pcl_rv-pEKEx2_fw
CGGCGTTTCACTTCTGAGTTCGGC

From a clone, which yielded a PCR product having the size 1.17 kb, a plasmid was prepared on a larger scale using the QIAGEN Plasmid Plus Maxi Kit (order no. 12963). The plasmid was labeled pCLTON2-bet, and the sequence thereof was labeled as SEQ ID No. 3.

### EXAMPLE 2b

### Production of pCLTON2-recT

To clone recT, the vector pRAC3 (Roles of RecJ, RecO, and RecR in RecET-mediated illegitimate recombination in Escherichia coli. Shiraishi K, Hanada K, Iwakura Y, Ikeda H, J Bacteriol. 2002 September; 184(17):4715-21) was isolated from E. coli using the QIAGEN Plasmid Plus Maxi Kit (order no. 12963). This plasmid served as a template for PCR amplification using the primer pairs recT-F and recT-R.

recT-F
aaggagatatagatATGACTAAGCAACCACCAATC
recT-R
CGGTTATTCCTCTGAATTATCG

The resulting fragment of 0.8 kb was isolated as described in Example 2a, ligated to pCLTON2 and used to transform E. coli DH5. Transformed cells were plated out onto 100 ng/ml spectinomycin-containing complex medium.

To test for desired ligation products, a colony PCR was carried out as described in Example 2a. From a clone, which yielded a PCR product having the size 1.194 kb, a plasmid was prepared on a larger scale. The plasmid was labeled pCLTON2-recT, and the sequence thereof was labeled as SEQ ID No. 4.

### EXAMPLE 2c

### Production of pCLTON2-Gp43

To clone gp43, the gene was synthesized by Eurofins-MWG-Operon (Anzingerstr. 7a, 85560 Ebersberg, Germany). The sequence of the synthesized fragment is indicated as SEQ ID No. 10. The fragment was prepared as a 1407 bp fragment using the restriction enzymes Bglll and EcoRI, treated with the Klenow fragment, and subsequently phosphorylated with T4 polynucleotide kinase from Fermentas (order no. EK0031). The fragment was isolated as described in Example 2a, ligated to pCLTON2 and used to transform E. coli DH5. Transformed cells were plated out onto 100 ng/ml spectinomycin-containing complex medium.

To test for desired ligation products, a colony PCR was carried out as described in Example 2a. From a clone, which yielded a PCR product having the size 1.79 kb, a plasmid was prepared on a larger scale. The plasmid was labeled pCLTON2-gp43, and the sequence thereof was labeled as SEQ ID No. 5.

### EXAMPLE 2d

### Production of pCLTON2-Gp61

To clone gp61, the gene was synthesized by Eurofins-MWG-Operon (Anzingerstr. 7a, 85560 Ebersberg, Germany). The sequence of the synthesized fragment is indicated as SEQ ID No. 11. The fragment was prepared as 1082 bp using the restriction enzymes BglII and MunI, treated with the Klenow fragment, and subsequently phosphorylated with T4 polynucleotide kinase from Fermentas (order no. EK0031). It was isolated as described in Example 2a, ligated to pCLTON2 and used to transform E. coli DH5. Transformed cells were plated out onto 100 ng/ml spectinomycin-containing complex medium.

To test for desired ligation products, a colony PCR was carried out as described in Example 2a. From a clone, which yielded a PCR product having the size 1.45 kb, a plasmid was prepared on a larger scale. The plasmid was labeled pCLTON2-gp61, and the sequence thereof was labeled as SEQ ID No. 6.

### EXAMPLE 2e

### Production of pCLTON2-rCAU

To clone rCau (cauri_1962), the gene was synthesized by Eurofins-MWG-Operon (Anzingerstr. 7a, 85560 Ebersberg, Germany). The sequence of the synthesized fragment is indicated as SEQ ID No. 1. The fragment was prepared as 839 bp using the restriction enzymes Bglll and MunI, treated with the Klenow fragment, and subsequently phosphorylated with T4 polynucleotide kinase from Fermentas (order no. EK0031). It was isolated as described in Example 2a, ligated to pCLTON2 and used to transform E. coli DH5. Transformed cells were plated out onto 100 ng/ml spectinomycin-containing complex medium.

To test for desired ligation products, a colony PCR was carried out as described in Example 2a. From a clone, which yielded a PCR product having the size 1.22 kb, a plasmid was prepared on a larger scale. The plasmid was labeled pCLTON2-rCau, and the sequence thereof was labeled as SEQ ID No. 7.

### EXAMPLE 2f

### Production of pEKEx3-recT

To clone recT in pEKEx3, pCLTON2-recT from Example 2b was used as a template for PCR amplification. The gene was amplified using the primer pairs BglII-RBS-RecT-F and EcoRI-RecT-R.

BglII-RBS-RecT-F
gcagatctaaggagatatacatATGACTAAGCAACCACCAATCG
EcoRI-RecT-R
gcgcgaattccaggCTGAATTATTCCTC

The resulting fragment of 0.84 kb was isolated by way of gel isolation using the Minielute Extraction Kit (order no. 28704) from Quiagen), treated with the Klenow fragment, and subsequently phosphorylated with T4 polynucleotide kinase from Fermentas (order no. EK003). The vector pEKEx3 was cut with EcoRI and BamHI, and the resulting fragment of 8298 bp was dephosphorylated using shrimp alkaline phosphatase from Fermentas (order no. EF0511). The fragment and the vector were ligated using the Rapid DNA Ligation Kit from Roche (order no. 11 635 379 001) and used to transform E. coli DH5. Transformed cells were plated out onto 100 ng/ml spectinomycin-containing complex medium.

To test for desired ligation products, a colony PCR was carried out using the primer pairs col-pEKEx3-F and col-pEKEx3-R.

coI-pEKEx3-F
CGCCGACATCATAACGGTTCTG
coI-pEKEx3-R
TTATCAGACCGCTTCTGCGTTC

From a clone, which yielded a PCR product having the size 1.71 kb, a plasmid was prepared on a larger scale using the QIAGEN Plasmid Plus Maxi Kit (order no. 12963). The plasmid was labeled pEKEx3-recT, and the sequence thereof was labeled as SEQ ID No. 8.

### EXAMPLE 2g

### Production of pEKEx3-Bet

To clone the recombinase Bet, pCLTON2-rCau from Example 2e was used as a template for PCR amplification. The gene was amplified using the primer pairs BglII-RBS-bet-F and EcoRI-bet-R.

BglII-RBS-bet-F
cggcagatctaaggagatatacatATGAGTACTGCACTCGCAAC
EcoRI-bet-R
gcgcggaattCATGCTGCCACCTTCTGC

The resulting fragment of 0.81 kb was isolated by way of gel isolation using the Minielute Extraction Kit (order no. 28704) from Quiagen), treated with the Klenow fragment, and subsequently phosphorylated with T4 polynucleotide kinase from Fermentas (order no. EK0031). The vector pEKEx3 was cut with EcoRI and BamHI, and the resulting fragment of 8298 bp was dephosphorylated using shrimp alkaline phosphatase from Fermentas (order no. EF0511). The fragment and the vector were ligated using the Rapid DNA Ligation Kit from Roche (order no. 11 635 379 001) and used to transform E. coli DH5. Transformed cells were plated out onto 100 ng/ml spectinomycin-containing complex medium.

To test for desired ligation products, a colony PCR was carded out as in Example 2e using the primer pairs col-pEKEx3-F and col-pEKEx3-r. From a clone, which yielded a PCR product having the size 1.08 kb, a plasmid was prepared on a larger scale using the QIAGEN Plasmid Plus Maxi Kit (order no. 12963). The plasmid was labeled pEKEx3-bet, and the sequence thereof was labeled as SEQ ID No. 9.

### EXAMPLE 3

### Production of a Test Strain

So as to insert a non-functional copy of a kanamycin resistance-imparting gene into the chromosome of C. glutamicum ATCC13032, initially the primer pairs ScaI-KanR-F/Kan(−)-L-R and MunI-R-R/Kan(−)-R-F were used to produce two PCR fragments to be fused as a template using the vector pJC1 (Cremer J, Treptow C, Eggeling L, Sahm H. Regulation of enzymes of lysine biosynthesis in Corynebacterium glutamicum. J Gen Microbiol. 1988; 134(12):3221-9).

ScaI-KanR-F
CGAGTACTACAAACGCGGCCATAAC
Kan(-)-L-R
GTCGGAAGAGGCATAGAATTCCGTCAGCCAGTTTAG
Kan(-)-R-F
GCTGACGGAATTCTATGCCTCTTCCGACCATC
MunI-R-R
ATACAATTGAACAAAGCCGCCGTCC

The two resulting PCR fragments were purified using the Minielute Extraction Kit (order no. 28704) from Quiagen and fused in a fusion PCR with the primer pairs ScaI-KanR-F/MunI-R-R to yield the defective kanamycin resistance gene. This includes a cytosine as an additional nucleotide in position 234, resulting in a frame shift such that the gene is not read completely. The resulting product was restricted using ScaO and Muni and subsequently cloned in the pK18mobsacB-lysOP7 cut in EcoRI and Seal (Acetohydroxyacid synthase, a novel target for improvement of L-lysine production by Corynebacterium glutamicum. Blombach B, Hans S, Bathe B, Eikmanns B J. Appl Environ Microbiol. 2009 January; 75(2):419-427). In this vector, the defective kanamycin resistance gene is flanked by two non-coding regions of the C. glutamicum genome, by way of which the homologous integration into the genome takes place. Thereafter, the entire cassette was integrated into the C. glutamicum genome between positions 1.045.503 and 1.045.596 using known methods by way of double positive selection (Small mobilizable multi-purpose cloning vectors derived from the Escherichia coli plasmids pK18 and pK19: selection of defined deletions in the chromosome of Corynebacterium glutamicum. Schafer A, Tauch A, Jäger W, Kalinowski J, Thierbach G, Pühler A Gene. 1994 Jul. 22; 145(1):69-73). The correct integration of the defective kanamycin resistance gene into the chromosome was checked using the primer pairs colNCR-L2 and colNCR-R2. The size of the PCR fragment was 3937 bp.

coINCR-L2:
CATTGGTCACCTTTGGCGTGTGG
coINCR-R2:
AATCAATGAGCGCCGTGAAGAAGG

### EXAMPLE 4

### Test for Recombinase Activity

The transformation of the test strain was carried out as described by Tauch et al. for Corynebacterium diphtheriae and C. glutamicum (Efficient electrotransformation of Corynebacterium diphtheriae with a mini-replicon derived from the Corynebacterium glutamicum plasmid pGC1. Tauch A, Kirchner O, LOffler B, Götker S, Pühler A, Kalinowski J. Curr Microbiol. 2002 November; 45(5):362-367). The strain was rendered competent, and in each case 0.5 micrograms of the vector coding for the recombinase was used for electroporation.

Spectinomycin-resistant clones were selected on the complex medium, brain heart infusion sorbitol, BHIS, (High efficiency electroporation of intact Corynebacterium glutamicum cells. Liebl W, Bayed A, Schein B, Stillner U, Schleifer K H. FEMS Microbiol Lett. 1989 December; 53(3):299-303), which contained 100 micrograms of spectinomycin (BHIS-Spec1OO). One clone each of the test strain containing the vector pCLTON2-bet, pCLTON2-recT, pCLTON2-gp43, pCLTON2-gp61, pCLTON2-rCau, pEKEx3-recT, or pEKEx3-bet was inoculated in 50 ml BHIS-Spec1OO and cultivated over night at 130 rpm and 30° C. in Erlenmeyer flasks. The next morning, 500 ml BHIS-Spec1OO+IPTG (0.5 mM when using pEKEx3-recT, pEKEx3-bet) or tetracycline (250 ng/ml when using pCLTON2-bet, pCLTON2-recT, pCLTON2-gp43, pCLTON2-gp61, pCLTON2-rCau) was inoculated with 10 ml of medium incubated overnight and cultivated for 4 to 6 hours until an OD of 1.5 to 2 was reached. Thereafter, the culture was cooled for 30 minutes on ice, washed twice with 50 ml 10% glycerol, 1 mM Tris pH 8, and subsequently twice with 10% glycerol. The cell pellet was 10% resuspended in the return flow and an additional 1 ml glycerol, aliquotted into 150 μl each, flash-frozen in liquid nitrogen, and stored at −75° C. until use. For use, the cells were gently thawed on ice within 20 minutes and mixed with 1 μg DNA.

The DNA used was the oligo Kan100*, having the sequence ATGCATCATCAGGAGTACGGATAAAATGCTTGATGGTCGGAAGAGGCATAAATTCCGTCAG CCAGTTTAGTCTGACCATCTCATCTGTAACATCATTGGC. This DNA was synthesized by Eurofins-MWG-Operon (Anzingerstr. 7a, 85560 Ebersberg, Germany).

The suspension of cells and 1 microgram DNA was transferred into electroporation cuvettes and carefully coated with 800 μl ice cold 10% glycerol and subsequently electroporated. For regeneration, the cells were immediately transferred into 4 ml of BHIS, which had been precontrolled to a temperature of 46° C., and incubated for 6 minutes at 46° C. Subsequently, a 1- to 6-hour cultivation was carried out at 30° C. and 170 rpm in a 15 ml flacon. Then the cells were transferred to BHIS, which contained 50 micrograms per milliliter of kanamycin. The result is shown in Table 1. It is apparent that, per batch, a maximum of 57 cells are spontaneously resistant to kanamycin; the maximum recombination frequency of 20054 clones was obtained with pEKEx3-recT; and decreasing recombinase activity occurs with pCLTON2-recT, pEKEx3-bet, pCLTON2-rCau, and pCLTON2-gp61. The recombinase activity of pCLTON2-gp43 is barely above background, and pCLTON2-bet is not active.

### EXAMPLE 5

### Optimization of Recombinase Activity

The test strain containing pEKEx3-recT was inoculated in 50 ml BHIS-Spec100 and cultivated overnight at 130 rpm and 30° C. in an Erlenmeyer flask. The next morning, 50 ml BHIS-Spec100+0.5 mM IPTG was inoculated with 10 ml of medium incubated overnight and cultivated for 0, 1, or 4 hours. The test strain with pCLTON2-recT was inoculated in 50 ml BHIS-Spec100 and cultivated overnight at 130 rpm and 30° C. in an Erlenmeyer flask. The next morning, 50 ml BHIS-Spec100+250 nanograms tetracycline was inoculated with 10 ml of medium incubated overnight and cultivated for 0, 1, or 4 hours. Thereafter, the culture was cooled for 30 minutes on ice, washed twice with 50 ml 10% glycerol, 1 mM Tris pH 8, and subsequently twice with 10% glycerol. The cell pellet was 10% resuspended in the return flow and an additional 1 ml glycerol, aliquotted into 150 μl each, flash-frozen in liquid nitrogen, and stored at −75° C. until use. For use, the cells were gently thawed on ice within 20 minutes and mixed with 1 microgram DNA.

The electroporation and regeneration were carried out as described in Example 4. Table 2 shows that the maximum recombination frequency is achieved in the vector pEKEx3-recT after 4 hours of induction when using the recombinase recT.

For further optimization, cells of the test strain containing pEKEx3-recT were used as previously, but increasing amounts of DNA were added. Table 3 shows that the maximum recombination frequency is achieved in the vector pEKE3-recT when using the recombinase recT at a concentration of 10 micrograms DNA.

### EXAMPLE 6

### Obtaining a Lysine Producer by Recombineering in the lysC Gene with a DNA Molecule

For the direct isolation of a strain producing increased amounts of lysine, starting from a starting strain, C. glutamicum ATCC13032 was transformed using the nanosensor pSenLys. The nanosensor pSenLys is described in WO2011138006. Cells of the resulting strain were transformed using pEKEx3-recT, and the recombinase was induced as described in Example 4. The DNA lysC-100* was synthesized by Eurofins-MWG-Operon (Anzingerstr. 7a, 85560 Ebersberg, Germany). It is stored as SEQ ID No. 32.

lysC-100*:
TCTTCAAGATCTCCATCGCGCGGCGGCCGTCGGAACGAGGGCAGGTGAA
GATGATATCGGTGGTGCCGTCTTCTACAGAAGAGACGTTCTGCAGAACC
AT

10 micrograms of the DNA lysC-100* were transferred into the strain by way of electroporation, as described in Example 4 (FIG. 1, C1). Thereafter, the strain was regenerated for 4 hours in BHIS with 100 microgram per milliliter of spectinomycin. The cells were then centrifuged and resuspended in 700 microliters CGXII glucose. This minimal medium is described by Keilhauer et al. (Isoleucine synthesis in Corynebacterium glutamicum: molecular analysis of the ilvB-ilvN-ilvC operon. Keilhauer C, Eggeling L, Sahm H. J Bacteriol. 1993 September; 175(17):5595-603). The cells were incubated for 40 hours at 30 degrees Celsius until the stationary phase was reached. Thereafter a 1:10 transfer was carried out into new CGXII glucose medium, followed by 4 hours of incubation, and the cell suspension was subjected to the cytometric product analysis and selection of individual cells (FIG. 2, C2).

For the flow cytometry analysis and sorting of the cells having high fluorescence, the cell suspension was adjusted in CGXII glucose medium to an optical density value of less than 0.1 and directly supplied to the ARIA II high-speed cell sorter (Becton Dickinson GmbH, Tullastr. 8-12, 69126 Heidelberg). The analysis was carried out using excitation wavelengths of 488 and 633 nm, and the detection was carried out at emission wavelengths of 530±15 nm and 660±10 nm at a test pressure of 70 psi. The data was analyzed by way of the software Version BD DIVA 6.1.3 associated with the device. Electronic gating was adjusted based on the forward and backward scatter so as to exclude non-bacterial particles. So as to sort EYFP-positive cells, the next stage of electronic gating was selected so as to exclude non-fluorescent cells. In this way, 51 fluorescent cells were sorted out on petri dishes containing BHIS medium.

The petri dish was incubated for 30 hours at 30 degrees Celsius, and subsequently each of the 46 reaction vessels of the microtiter plate Flowerplate (48-well) of the BioLector cultivation system (m2plabs GmbH, Aachen, Germany) was inoculated with a respective clone. Each reaction vessel contained 0.7 microliters CGXII glucose. One of the reaction vessels was inoculated with a negative control, and one was inoculated with a positive control. Thereafter, the microtiter plate was incubated for 2 days at 30° C., 1200 rpm, and a shaking radius of 3 mm. In the BioLector cultivation system, the growth was recorded online as scattered light at 620 nm, and the fluorescence of the culture was recorded continuously at an excitation wavelength of 485 nm and an emission wavelength of 520 nm.

After 2 days, the specific fluorescence of the cultures was determined based on the recorded data. It was elevated in 33 clonal cultures at least four-fold compared to the negative control. The lysC sequence in the genome was determined in 12 of these cultures. In all instances, the cytosine in position 932 of the gene had been exchanged with a thymidine. The sequence thus corresponded to the sequence part that was present on the synthesized oligo lysC-100* and results in the lysine formation with C. glutamicum (Binder et al. Genome Biology 2012, 13:R40).

### EXAMPLE 7

### Obtaining a Lysine Producer by Recombineering in the murE Gene with Multiple DNA Molecules Simultaneously

For the direct isolation of a strain producing increased amounts of lysine, starting from a starting strain using murE mutations, the starting strain C. glutamicum ATCC13032 described in Example 6 was used with pSenLys and pEKEx3-recT. The individual murE DNA oligos were synthesized by Eurofins-MWG-Operon (Anzingerstr. 7a, 85560 Ebersberg, Germany). The following murE sequences were used: murEG81amb*, SEQ ID No. 12; murEG81A*, SEQ ID No. 13; murEG81C*, SEQ ID No. 14; murE G81D*, SEQ ID No. 15; murEG81E*, SEQ ID No. 16; murEG81F*, SEQ ID No. 17; murEG81H*, SEQ ID No. 18; murEG81I*, SEQ ID No. 19; murEG81K*, SEQ ID No. 20; murEG81L*, SEQ ID No. 21; murEG81M*, SEQ ID No. 22; murEG81N*, SEQ ID No. 23; murEG81P*, SEQ ID No. 24; murEG81Q*, SEQ ID No. 25; murEG81R*, SEQ ID No. 26; murEG81S*, SEQ ID No. 27; murEG81T*, SEQ ID No. 28; murEG81V*. SEQ ID No. 29; murEG81W*, SEQ ID No. 30; murEG81Y*, SEQ ID No. 31.

1 microgram of these DNA oligos was removed in each case, and the resulting 20 micrograms were mixed with an aliquot of cells and transferred in the strain by way of electroporation, as described in Example 4 (FIG. 1, C1). Thereafter, the regeneration of the cells, with the subsequent cultivations and flow cytometry analysis and sorting of the cells (FIG. 2, C2) were carried out, as described in Example 5.

In this way, 62 fluorescent cells were sorted out on petri dishes containing BHIS medium. The petri dish was incubated for 30 hours at 30 degrees Celsius, and subsequently each of the 46 reaction vessels of the microtiter plate Flowerplate (48-well) of the BioLector cultivation system (m2plabs GmbH, Aachen, Germany) was inoculated with a respective clone. Each reaction vessel contained 0.7 microliters CGXII glucose. One of the reaction vessels was inoculated with a negative control, and one was inoculated with a positive control. Thereafter, the microtiter plate was incubated for 2 days at 30° C., 1200 rpm, and a shaking radius of 3 mm. In the BioLector cultivation system, the growth was recorded online as scattered light at 620 nm, and the fluorescence of the culture was recorded continuously at an excitation wavelength of 485 nm and an emission wavelength of 520 nm.

After 2 days, the specific fluorescence of the cultures was determined based on the recorded data. It was elevated in 33 clonal cultures at least twelve-fold compared to the negative control. An L-lysine determination in the medium was carried out for 21 cultures to verify the product formation (FIG. 2, C3). The lysine determination was carried out as o-phthaldialdehyde derivative by way of high-pressure liquid chromatography using a uHPLC 1290 Infinity system (Agilent) on a Zorbax Eclipse AAA C18 3.5 micron 4.6×75 mm reversed-phase column and a fluorescence detector. The eluent used was a gradient of 0.01 M Na borate pH 8.2 with increasing methanol concentration, and the detection of the fluorescent isoindole derivatives was carried out at an excitation wavelength of 230 nm and an emission wavelength of 450 nm. The L-lysine values shown in Table 4 were determined, which show an improvement in the L-lysine production compared to the starting strain.

The murE sequence in the genome was determined for these 21 clones. Sequencing was carried out after PCR amplification by the company Eurofins-MWG-Operon (Anzingerstr. 7a, 85560 Ebersberg, Germany). The resulting mutations are summarized in Table 4. It is apparent that in this way 10 different murE mutations were obtained starting from the starting strain, of which nine resulted in increased lysine formation compared to the starting strain. The sequences of the murE alleles obtained are SEQ ID No. 33, murEG81W; SEQ ID No. 34, murEG81Y; SEQ ID No. 35, murEG81N; SEQ ID No. 36, murEG81C; SEQ ID No. 37, murEG81S; SEQ ID No. 38, murEG81F; SEQ ID No. 39, murEG81V; SEQ ID No. 40, murEG81L; SEQ ID No. 41, murEG81H; SEQ ID No. 42, murEG81I; SEQ ID No. 43, murEG81T; and SEQ ID No. 44, murEG81R.

TABLE 1
Comparison of recombinase activities in Corynebacterium glutamicum

| | cfu^{a} | cfu |
|---|---|---|
| Vector | (rec) | (spont) |
| pCLTON2-bet | 8 | 0 |
| pCLTON2-recT | 12513 | 31 |
| pCLTON2-gp43 | 97 | 57 |
| pCLTON2-gp61 | 306 | 1 |
| pCLTON2-rCau | 2475 | 7 |
| pEKEx3-recT | 20054 | 44 |
| pEKEx3-bet | 6491 | 12 |

^{a}cfu (rec) indicates the number of kanamycin-resistant clones that resulted from recombineering with the Kan* oligo; cfu (spont) is the number of spontaneously kanamycin-resistant clones that resulted from a control batch, which contained water instead of the Kan* oligos. It is clearly apparent that high recombination efficiency is achieved with the recombinase recT in pEKEx3 recT or pCLTON2-recT. Very high recombination efficiency is also achieved with the recombinase rCau from Corynebacterium aurimucosum, which clearly exceeds that of the spontaneously resistant clones.

TABLE 2
Comparison of recombinase activities after varying induction times

| | Induction time | cfu^{a} | cfu |
|---|---|---|---|
| Vector | (h) | (rec) | (spont) |
| pCLTON2-recT | 0 | 101 | 2 |
| pCLTON2-recT | 1 | 816 | 1 |
| pCLTON2-recT | 4 | 8831 | 6 |
| pEKEx3-recT | 0 | 1238 | 6 |
| pEKEx3-recT | 1 | 53460 | 7 |
| pEKEx3-recT | 4 | 33165 | 7 |

^{a}cfu (rec) and cfu (spont) are the same as in Table 1. The influence of the induction time for expression of the recombinase on the recombination efficiency is clearly apparent.

TABLE 3
Comparison of recombinase activities using varying DNA amounts

| | Amount | cfu |
|---|---|---|
| Vector | μ(g) | (rec) |
| pEKEx3-recT | 0 | 13 |
| pEKEx3-recT | 0.05 | 1139 |
| pEKEx3-recT | 0.1 | 2673 |
| pEKEx3-recT | 0.5 | 25080 |
| pEKEx3-recT | 1 | 15840 |
| pEKEx3-recT | 5 | 301950 |
| pEKEx3-recT | 10 | 950400 |
| pEKEx3-recT | 25 | 940500 |
| pEKEx3-recT | 50 | 871200 |
| pEKEx3-recT | 100 | 831600 |

^{a}cfu (rec) and cfu (spont) are the same as in Table 1. It is clearly apparent how the DNA amount added to the recombineering batch increases the recombineering frequency. The maximum recombineering frequency is reached at approximately 10 micrograms DNA.

TABLE 4
Results of the sequencing of murE alleles in clones that were
obtained by way of recombineering and direct cytometric
product analysis by the nanosensor (FIG. 3) and after
verification (FIG. 3, C3.B), and the lysine formation and
fluorescence of the same in cultures.

| | murE | | | |
|---|---|---|---|---|
| | Codone | MurE amino acid | Fluorescence | Lysine |
| Strain | 241-243 | 81 | (AU) | (mM) |
| Starting strain | GGA | (G) glycine | 0.07 | 0 |
| I.4 | TGG | (W) tryptophan | 1.80 | 11 |
| I.6 | TAC | (Y) tyrosine | 1.17 | 9 |
| I.7 | AAC | (N) asparagine | 0.73 | 5 |
| I.24 | TTC | (F) phenylalanine | 1.36 | 8 |
| I.25 | TGC | (C) cysteine | 1.08 | 7 |
| I.34 | CTG | (L) leucine | 1.83 | 12 |
| II.1 | CAC | (H) histidine | 0.46 | 1 |
| II.4 | GTG | (V) valine | 1.47 | 9 |
| II.5 | ACC | (T) threonine | 0.47 | 1 |
| II.24 | ATC | (I) isoleucine | 1.85 | 10 |
| II.23 | CGC | (R) arginine | 2.05 | 10 |

Sequences according to sequence listing:

| SEQ ID | Name |
|---|---|
| SEQ ID No. 1 | recombinase gene |
| SEQ ID No. 2 | recombinase |
| SEQ ID No. 3 | pCLTON2-bet |
| SEQ ID No. 4 | pCLTON2-recT |
| SEQ ID No. 5 | pCLTON2-gp43 |
| SEQ ID No. 6 | pCLTON2-gp61 |
| SEQ ID No. 7 | pCLTON2-rCau |
| SEQ ID No. 8 | pEKEx3-recT |
| SEQ ID No. 9 | pEKEx3-bet |
| SEQ ID No. 10 | gp43 adapted |
| SEQ ID No. 11 | gp61 adapted |
| SEQ ID No. 12 | murEG81amb* |
| SEQ ID No. 13 | murEG81A* |
| SEQ ID No. 14 | murEG81C* |
| SEQ ID No. 15 | murEG81D* |
| SEQ ID No. 16 | murEG81E* |
| SEQ ID No. 17 | murEG81F* |
| SEQ ID No. 18 | murEG81H* |
| SEQ ID No. 19 | murEG81I* |
| SEQ ID No. 20 | murEG81K* |
| SEQ ID No. 21 | murEG81L* |
| SEQ ID No. 22 | murEG81M* |
| SEQ ID No. 23 | murEG81N* |
| SEQ ID No. 24 | murEG81P* |
| SEQ ID No. 25 | murEG81Q* |
| SEQ ID No. 26 | murEG81R* |
| SEQ ID No. 27 | murEG81S* |
| SEQ ID No. 28 | murEG81T* |
| SEQ ID No. 29 | murEG81V* |
| SEQ ID No. 30 | murEG81W* |
| SEQ ID No. 31 | murEG81Y* |
| SEQ ID No. 32 | lysC-100* |
| SEQ ID No. 33 | murEG81W |
| SEQ ID No. 34 | murEG81Y |
| SEQ ID No. 35 | murEG81N |
| SEQ ID No. 36 | murEG81C |
| SEQ ID No. 37 | murEG81S |
| SEQ ID No. 38 | murEG81F |
| SEQ ID No. 39 | murEG81V |
| SEQ ID No. 40 | murEG81L |
| SEQ ID No. 41 | murEG81H |
| SEQ ID No. 42 | murEG81I |
| SEQ ID No. 43 | murEG81T |
| SEQ ID No. 44 | murEG81R |

1. A microorganism that is genetically modified compared to the wild type thereof, comprising a gene sequence coding for a recombinase not present in the wild type and furthermore a gene sequence coding for a metabolite sensor.

2. The cell microorganism according to claim 1, wherein the gene sequence coding for a metabolite sensor is a sequence coding for a protein that detects an amino acid, organic acid, fatty acid, vitamin, or a plant active agent.

3. The microorganism according to claim 1, wherein the gene sequence coding for a recombinase is a sequence coding for a protein that recombines extracellularly added DNA with intracellular DNA.

4. (canceled)

5. A microorganism according to claim 1, wherein the microorganism is a microorganism of the genus Corynebacterium, Enterobacterium or Escherichia.

6.-20. (canceled)

21. A method for identifying a microorganism from the group consisting of Corynebacterium, Enterobacterium or Escherichia, containing a vector according to sequence 4 or 8, having an intracellular concentration of a particular metabolite that is increased compared to the wild type of the microorganism from the group consisting of amino acids, organic acids, fatty acids, vitamins, or plant active agents in a cell suspension, comprising the following method steps:
i) providing a cell suspension including the microorganism from the group consisting Corynebacterium, Enterobacterium or Escherichia that contains a vector according to sequence 4 or 8 and additionally contains a gene sequence that codes for a metabolite sensor and codes for a metabolite sensor, which detects metabolites from the group consisting of amino acids, organic acids, fatty acids, vitamins or plant active agents;
ii) genetically modifying the cells according to step i) by recombineering while adding DNA that contains at least one modified gene G1 to Gn, or at least one mutation M1 to Mm, obtaining a cell suspension in which the cells differ in terms of the intracellular concentration of the metabolite; and
iii) identifying individual cells in the cell suspension having an increased intracellular concentration of the metabolite by fluorescence detection using a metabolite sensor for amino acids, organic acids, fatty acids, vitamins or plant active agents.

22. A method for producing a microorganism that is genetically modified compared to the wild type thereof from the group consisting of Corynebacterium, Enterobacterium or Escherichia, having optimized production of a metabolite from the group consisting of amino acids, organic acids, fatty acids, vitamins, or plant active agents, comprising the following method steps:
i) providing a cell suspension including the microorganism from the group consisting Corynebacterium, Enterobacterium or Escherichia that contains a vector according to sequence 4 or 8 and additionally contains a gene sequence that codes for a metabolite sensor and codes for a metabolite sensor, which detects metabolites from the group consisting of amino acids, organic acids, fatty acids, vitamins or plant active agents;
ii) genetically modifying the cells according to step i) by recombineering while adding DNA that contains at least one modified gene G1 to Gn, or at least one mutation M1 to Mm, obtaining a cell suspension in which the cells differ in terms of the intracellular concentration of a particular metabolite;
iii) identifying individual cells in the cell suspension having an increased intracellular concentration of the metabolite by fluorescence detection using a metabolite sensor for amino acids, organic acids, fatty acids, vitamins or plant active agents.
iv) separating the identified cells from the cell suspension;
v) identifying at least one genetically modified gene G1 to Gn, or at least one mutation M1 to Mm, in the identified and separated cells that are responsible for the increased intracellular concentration of the metabolite; and
vi) producing a production cell that is genetically modified compared to the wild type thereof and has optimized production of the metabolite, the genome of the metabolite comprising at least one of the genes G1 to Gm and/or at least one mutation M1 to Mm.

23. A method according to claim 21, wherein the genetic modification of the cell according to step ii) is carried out by a recombinase, which inserts one or more DNA molecules that are introduced into the cell and contain the modified gene or the modified genes G1 to Gn and/or the mutation or the mutations M1 to Mm into the intracellular DNA, which is present as a chromosome or plasmid.

24. A method according to claim 22, wherein DNA is used for at least one modified gene G1 to Gn and/or at least one mutation M1 to Mm, which code for one of the steps from the biosynthesis pathway of the metabolite.

25. A method for producing metabolites, comprising the following method steps:
a) producing a cell that is genetically modified compared to the wild type thereof and has optimized production of a particular metabolite using a method according to claim 22, and
b) cultivating the cell in a culture medium containing nutrients under conditions in which the cell produces the particular metabolite from the nutrients.

26. The method according to claim 25, wherein the metabolite is a component from the group consisting of amino acids, organic acids, fatty acids, vitamins, or plant active agents.
